(19) United States
(12) Patent Application Publication
(10) Pub. No.: US 2025/0161260 A1
(43) Pub. Date: May 22, 2025

(54) USE OF DELTA-TOCOTRIENOL TO REDUCE THE PROGRESSION OF A NEOPLASM TO A CANCER

(71) Applicant: H. LEE MOFFITT CANCER CENTER AND RESEARCH INSTITUTE, INC., Tampa, FL (US)

(72) Inventor: Mokenge MALAFA, Tampa, FL (US)

(21) Appl. No.: 18/833,013

(22) PCT Filed: Jan. 27, 2023
(86) PCT No.: PCT/US2023/011783

§ 371 Date: Jul. 25, 2024

Related U.S. Application Data
(60) Provisional application No. 63/303,810, filed on Jan. 27, 2022.

(51) Int. Cl. A61K 31/355; A61K 31/616; A61P 35/00

(52) U.S. Cl. CPC A61K 31/355; A61K 31/616; A61P 35/00

(57) ABSTRACT

Disclosed are compositions comprising delta-tocotrienol and methods for using the same in the prevention of cancer progression. The methods comprise of reducing, inhibiting, decreasing, and/or preventing the progression of a neoplasm to a cancer (including, but not limited to lung cancer, ovarian cancer, breast cancer, rectal cancer, pancreatic, colon cancer, or intraductal papillary mucinous neoplasm (IPMN) to pancreatic ductal adenocarcinoma) in a subject comprising administering to a subject a therapeutically effective amount of a composition comprising delta-tocotrienol (d-T3).

This application claims the benefit of U.S. Provisional Application No. 63/303,810, filed on Jan. 27, 2022, which is incorporated herein by reference in its entirety.

### I. BACKGROUND

Pancreatic ductal adenocarcinoma (PDA) is an incalcitrant malignancy with a 5-year survival of only 10%. Therefore, targeting PDA precursors is an area of the highest priority. Amongst the most prevalent and easily identifiable high-risk individuals with PDA precursors are patients with intraductal papillary mucinous neoplasm (IPMN). Currently, patients with IPMN are managed with active surveillance (AS) and surgery to develop high-risk stigmata. FDA-approved non-operative treatment of IPMN to prevent progression to PDA does not exist. What is needed are new treatments that can prevent progression of neoplasms to cancer.

### II. SUMMARY

Disclosed are methods and compositions related to the use of composition comprising δ-tocotrienol to prevent cancer progression.

In one aspect, this application discloses methods of reducing, inhibiting, decreasing, and/or preventing the progression of a neoplasm to a cancer (including, but not limited to lung cancer, ovarian cancer, breast cancer, rectal cancer, pancreatic, colon cancer, or intraductal papillary mucinous neoplasm (IPMN) to pancreatic ductal adenocarcinoma) in a subject comprising administering to a subject a therapeutically effective amount of a composition comprising δ-tocotrienol (d-T3) (such as for example, a composition comprising a dose of d-T3 between about 400 mg and 800 mg).

The method of inhibiting progression of any preceding aspect, wherein the cancer progression being inhibiting is progression occurring following surgical removal or anti-cancer treatment of a cancer.

In one aspect the composition can be greater than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% d-T3.

In some aspects the d-T3 can be comprised of D-amino acids or L-amino acids or other synthetic variants of d-T3.

The tocotrienol for the aspects disclosed herein has the formula:

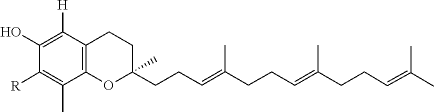

Wherein R is selected from the group consisting of H and CH₃.

In one aspect, disclosed herein are methods of inhibiting progression of a neoplasm to a cancer recurrence of any preceding aspect, further comprising administering to the subject aspirin.

### III. BRIEF DESCRIPTION OF THE DRAWINGS

The accompanying drawings, which are incorporated in and constitute a part of this specification, illustrate several embodiments and together with the description illustrate the disclosed compositions and methods.

FIGS. 1A and 1B show that DT3 significantly induced apoptosis as measured by cleaved caspase-3 expression (IHC) in patients with IPMN. FIG. 1A shows quantitative IHC analyses of cleaved caspase-3 levels in patients treated with DT 3, (% positive cells) in adjacent normal pancreatic ducts (N), invasive PDA (PA), IPMNs (IP), PanIN (IP), and Mucinous Cystic Neoplasia (MU). FIG. 1B shows representative IHC sections from pancreatectomy specimens stained for cleaved caspase—from patient 9 (300 mg orally twice daily dose level) who displayed bioactivity (defined as significant induction of apoptosis) to DT 3 treatment and patient 10 (400 mg orally twice daily dose level) who did not ‘respond’ to DT 3 treatment. Lower panel is IHC stained for the pro-apoptotic protein Bax, illustrating the association of DT 3 induction of apoptosis with increased expression of Bax

FIGS. 2A and 2B show the expression of MUC4 in IPMNs. In FIG. 2A, MUC4 expression is higher in the intestinal type (left) compared to the gastric type (right) IPMNs. In FIG. 2B, MUC4, when expressed in gastric-type IPMNs is restricted to regions of high-grade dysplasia (red arrow) but undetectable in areas of not low-grade dysplasia (black arrow).

FIG. 3 shows Expression of MUC4 during longitudinal follow up of IPMN lesions. IPMN lesions from 74-year old female who initially underwent distal pancreatectomy and splenectomy for IPMN with invasive carcinoma and then two years later developed a second tumor in the head of the pancreas and underwent complete pancreatectomy (Whipple procedure). Expression of MUC4 in: Panel A: Low-grade lesions; Panel B: Mixed low-grade and high-grade lesions; Panel C: high-grade lesions and D: Invasive PDA tumor. MUC4 expression was restricted to high-grade IPMN lesion (red arrow in panel B) while no expression was observed in low-grade area of the same lesions (blue arrow panel B). MUC4 expression progressively increased with the disease progression (unpublished data by Bouvet and Batra).

FIG. 4 shows Expression of MUC4 in the resected tissues from the responder (patient 4) and non-responder (patient 10) following DT3 treatment during in Phase 1 trial. Response was defined by positivity for cleaved caspase 3 or BAX (as detailed in FIG. 1)

FIGS. 5A and 5B show the top differentially expressed genes associated with various cell death pathways in responders following DT 3 treatment (5A). FIG. 5B indicates the pathway network associated with this gene signature.

FIG. 6 shows Ki67 staining for tumor cell proliferation showed increased tumor cell proliferation in KC-MUC4 (C, D) compared to KC (A, B) mice.

FIGS. 7A, 7B, 7C, and 7D show that DT3 suppresses Wnt/p-catenin activity in 3T3 cells, HPNE-Kras and PCSCs. FIG. 7A shows that in 3T3 cells, Wnt receptor activity (luciferase units) is induced by agonist (Wnt3a) and inhibited when Wnt3a treatment is followed by the Wnt antagonist (DKK1). DT 3 treatment significantly inhibited the agonist-induced receptor activity. FIG. 7B shows Wnt receptor agonist treatment induced β-catenin expression in 3T3 cells, which was inhibited by the Wnt antagonists DKK1 and DT 3. FIG. 7C shows treatment with Wnt antagonist DKK1 and DT 3 abolished β-catenin expression in HPNE-Kras cells and human PC stem cells (PCSCs), although PCSCs that were not pretreated with Wnt agonist Wnt3a had less inhibition of β-catenin expression with DT 3 treatment. FIG. 7D shows DT 3 treatment selectively induced apoptosis (C-PARP) in HPNE-Kras cells but not in HPNE cells and decreased the expression of MUC4 and MUC16 as well as β-catenin and its downstream signaling proteins (C-myc, cyclin D1 and survivin). DT 3 also inhibited pAKT and pERK, which are well-established downstream signaling proteins of oncogenic KRAS.

FIG. 8 shows that DT3 inhibits the proliferation of IPMN cell lines. LGKC1 (left) and LGKC2 cells were treated with varying doses of DT3 for 24, 48, and 72 h and cell proliferation were measured by MTT assay.

FIGS. 9A, 9B, 9C, 9D, 9E, and 9F show that DT3 induces apoptosis and induces cell cycle arrest in IPMN cell lines. LGKC1 (9A, 9C, 9E) and LGKC2 (9B, 9D, 9E) cells were treated with IC50 dose of DT 3 for 48 h. Apoptosis was measured by flow cytometry following annexin V and propidium iodide staining (9A, 9B). For cell cycle analysis by flow cytometry (9C, 9D) cells were synchronized with double thymidine block prior to treatment with DT 3. Following the treatment, cell lysates were prepared and impact on downstream signaling was performed by immunoblotting using the indicated antibodies (9E) and subjected to RNA sequencing (9F). Scatter plot depicting upregulated Gene Ontology Biological Processes of the RNAseq analysis of DT 3-treated cell lines.

FIGS. 10A, 10B, 10C, and 10D show the progression, percentage, and grade of PanINs and IPMNs of in KC, KCMUC4 and KPC mouse models (H&E staining). Significantly high penetrance of PanINs/IPMN lesions in KC-MUC4T G mice compared to KC and KPC mice. FIGS. 10A and 10B show PanIN/IPMN percentage at 5 and 30 weeks of PC progression KC, KCMUC4 and KPC mouse models (10C and 10D) PanIN/IPMN grade at 5 and 30 weeks of PC KC, KCMUC4 and KPC mouse models.

### IV. DETAILED DESCRIPTION

Before the present compounds, compositions, articles, devices, and/or methods are disclosed and described, it is to be understood that they are not limited to specific synthetic methods or specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

### A. Definitions

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

An “increase” can refer to any change that results in a greater amount of a symptom, disease, composition, condition or activity. An increase can be any individual, median, or average increase in a condition, symptom, activity, composition in a statistically significant amount. Thus, the increase can be a 1, 2, 3, 4, 5, 6, 7, 8, 9, 10, 15, 20, 25, 30, 35, 40, 45, 50, 55, 60, 65, 70, 75, 80, 85, 90, 95, or 100% increase so long as the increase is statistically significant.

A “decrease” can refer to any change that results in a smaller amount of a symptom, disease, composition, condition, or activity. A substance is also understood to decrease the genetic output of a gene when the genetic output of the gene product with the substance is less relative to the output of the gene product without the substance. Also for example, a decrease can be a change in the symptoms of a disorder such that the symptoms are less than previously observed. A decrease can be any individual, median, or average decrease in a condition, symptom, activity, composition in a statistically significant amount. Thus, the decrease can be a 1, 2, 3, 4, 5, 6, 7, 8, 9, 10, 15, 20, 25, 30, 35, 40, 45, 50, 55, 60, 65, 70, 75, 80, 85, 90, 95, or 100% decrease so long as the decrease is statistically significant.

“Inhibit,” “inhibiting,” and “inhibition” mean to decrease an activity, response, condition, disease, or other biological parameter. This can include but is not limited to the complete ablation of the activity, response, condition, or disease. This may also include, for example, a 10% reduction in the activity, response, condition, or disease as compared to the native or control level. Thus, the reduction can be a 10, 20, 30, 40, 50, 60, 70, 80, 90, 100%, or any amount of reduction in between as compared to native or control levels.

By “reduce” or other forms of the word, such as “reducing” or “reduction,” is meant lowering of an event or characteristic (e.g., tumor growth). It is understood that this is typically in relation to some standard or expected value, in other words it is relative, but that it is not always necessary for the standard or relative value to be referred to. For example, “reduces tumor growth” means reducing the rate of growth of a tumor relative to a standard or a control.

By “prevent” or other forms of the word, such as “preventing” or “prevention,” is meant to stop a particular event or characteristic, to stabilize or delay the development or progression of a particular event or characteristic, or to minimize the chances that a particular event or characteristic will occur. Prevent does not require comparison to a control as it is typically more absolute than, for example, reduce. As used herein, something could be reduced but not prevented, but something that is reduced could also be prevented. Likewise, something could be prevented but not reduced, but something that is prevented could also be reduced. It is understood that where reduce or prevent are used, unless specifically indicated otherwise, the use of the other word is also expressly disclosed.

The term “subject” refers to any individual who is the target of administration or treatment. The subject can be a vertebrate, for example, a mammal. In one aspect, the subject can be human, non-human primate, bovine, equine, porcine, canine, or feline. The subject can also be a guinea pig, rat, hamster, rabbit, mouse, or mole. Thus, the subject can be a human or veterinary patient. The term “patient” refers to a subject under the treatment of a clinician, e.g., physician.

The term “therapeutically effective” refers to the amount of the composition used is of sufficient quantity to ameliorate one or more causes or symptoms of a disease or disorder. Such amelioration only requires a reduction or alteration, not necessarily elimination.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder.

“Biocompatible” generally refers to a material and any metabolites or degradation products thereof that are generally non-toxic to the recipient and do not cause significant adverse effects to the subject.

“Comprising” is intended to mean that the compositions, methods, etc. include the recited elements, but do not exclude others. “Consisting essentially of” when used to define compositions and methods, shall mean including the recited elements, but excluding other elements of any essential significance to the combination. Thus, a composition consisting essentially of the elements as defined herein would not exclude trace contaminants from the isolation and purification method and pharmaceutically acceptable carriers, such as phosphate buffered saline, preservatives, and the like. “Consisting of” shall mean excluding more than trace elements of other ingredients and substantial method steps for administering the compositions provided and/or claimed in this disclosure. Embodiments defined by each of these transition terms are within the scope of this disclosure.

A “control” is an alternative subject or sample used in an experiment for comparison purposes. A control can be “positive” or “negative.”

“Effective amount” of an agent refers to a sufficient amount of an agent to provide a desired effect. The amount of agent that is “effective” will vary from subject to subject, depending on many factors such as the age and general condition of the subject, the particular agent or agents, and the like. Thus, it is not always possible to specify a quantified “effective amount.” However, an appropriate “effective amount” in any subject case may be determined by one of ordinary skill in the art using routine experimentation. Also, as used herein, and unless specifically stated otherwise, an “effective amount” of an agent can also refer to an amount covering both therapeutically effective amounts and prophylactically effective amounts. An “effective amount” of an agent necessary to achieve a therapeutic effect may vary according to factors such as the age, sex, and weight of the subject. Dosage regimens can be adjusted to provide the optimum therapeutic response. For example, several divided doses may be administered daily or the dose may be proportionally reduced as indicated by the exigencies of the therapeutic situation.

A “pharmaceutically acceptable” component can refer to a component that is not biologically or otherwise undesirable, i.e., the component may be incorporated into a pharmaceutical formulation provided by the disclosure and administered to a subject as described herein without causing significant undesirable biological effects or interacting in a deleterious manner with any of the other components of the formulation in which it is contained. When used in reference to administration to a human, the term generally implies the component has met the required standards of toxicological and manufacturing testing or that it is included on the Inactive Ingredient Guide prepared by the U.S. Food and Drug Administration.

“Pharmaceutically acceptable carrier” (sometimes referred to as a “carrier”) means a carrier or excipient that is useful in preparing a pharmaceutical or therapeutic composition that is generally safe and non-toxic and includes a carrier that is acceptable for veterinary and/or human pharmaceutical or therapeutic use. The terms “carrier” or “pharmaceutically acceptable carrier” can include, but are not limited to, phosphate buffered saline solution, water, emulsions (such as an oil/water or water/oil emulsion) and/or various types of wetting agents. As used herein, the term “carrier” encompasses, but is not limited to, any excipient, diluent, filler, salt, buffer, stabilizer, solubilizer, lipid, stabilizer, or other material well known in the art for use in pharmaceutical formulations and as described further herein.

“Pharmacologically active” (or simply “active”), as in a “pharmacologically active” derivative or analog, can refer to a derivative or analog (e.g., a salt, ester, amide, conjugate, metabolite, isomer, fragment, etc.) having the same type of pharmacological activity as the parent compound and approximately equivalent in degree.

“Therapeutic agent” refers to any composition that has a beneficial biological effect. Beneficial biological effects include both therapeutic effects, e.g., treatment of a disorder or other undesirable physiological condition, and prophylactic effects, e.g., prevention of a disorder or other undesirable physiological condition (e.g., a non-immunogenic cancer). The terms also encompass pharmaceutically acceptable, pharmacologically active derivatives of beneficial agents specifically mentioned herein, including, but not limited to, salts, esters, amides, proagents, active metabolites, isomers, fragments, analogs, and the like. When the terms “therapeutic agent” is used, then, or when a particular agent is specifically identified, it is to be understood that the term includes the agent per se as well as pharmaceutically acceptable, pharmacologically active salts, esters, amides, proagents, conjugates, active metabolites, isomers, fragments, analogs, etc.

“Therapeutically effective amount” or “therapeutically effective dose” of a composition (e.g. a composition comprising an agent) refers to an amount that is effective to achieve a desired therapeutic result. In some embodiments, a desired therapeutic result is the control of type I diabetes. In some embodiments, a desired therapeutic result is the control of obesity. Therapeutically effective amounts of a given therapeutic agent will typically vary with respect to factors such as the type and severity of the disorder or disease being treated and the age, gender, and weight of the subject. The term can also refer to an amount of a therapeutic agent, or a rate of delivery of a therapeutic agent (e.g., amount over time), effective to facilitate a desired therapeutic effect, such as pain relief. The precise desired therapeutic effect will vary according to the condition to be treated, the tolerance of the subject, the agent and/or agent formulation to be administered (e.g., the potency of the therapeutic agent, the concentration of agent in the formulation, and the like), and a variety of other factors that are appreciated by those of ordinary skill in the art. In some instances, a desired biological or medical response is achieved following administration of multiple dosages of the composition to the subject over a period of days, weeks, or years.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### B. Methods of Inhibiting Progression of a Neoplasm to a Cancer

Disclosed are the components to be used to prepare the disclosed compositions as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a particular δ-tocotrienol (d-T3) is disclosed and discussed and a number of modifications that can be made to a number of molecules including the δ-tocotrienol (d-T3) are discussed, specifically contemplated is each and every combination and permutation of d-T3 and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

d-T3, found in nuts, grains, palm oil, and other plant materials, having the formula 1 below contains a lipophilic side chain with 16 carbons and 3 double bonds, as well as a chromanol ring with a phenolic group at position 6 and a methyl group at position 8.

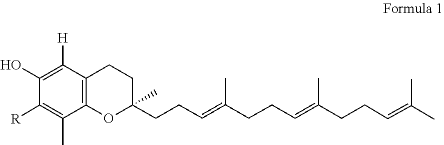

Wherein R is selected from the group consisting of H and CH₃.

The phenolic group provides antioxidant activities. With the 5 group unmethylated, d-T3 has also strong activities in quenching reactive nitrogen species. Although a member of the vitamin E family, d-T3 has properties different from the commonly studied α-tocopherol (a-T), which has no double bond on the side chain and is trimethylated at positions 5, 7, and 8 of the chromanol ring. The structural difference makes these two compounds very different in cancer preventive activities. With inconsistent results in laboratory studies, clinical trials with large doses of a-T yielded disappointing results. On the other hand, d-T3 has shown promising cancer preventive effects. In terms of the mechanisms, most studies have reported inhibition of the Wnt pathway and activation of apoptosis in colon cancer; however, the primary targets remain to be identified. Of note is the reported inhibition of COX-1/2, 15-LOX, and dihydroceramide desaturase by d-T3/metabolites. The results also indicate that induction of apoptosis through β-catenin degradation can also be an important mechanism of d-T3 chemopreventive activity. In addition to the above-mentioned mechanisms, the quenching of reactive oxygen and nitrogen species and the activities of the side-chain degradation metabolites can be important. Because d-T3 is not effectively transported from the liver to the blood by a-T transport protein, the systemic bioavailability of d-T3 is much lower than a-T. The d-T3 in the liver undergoes side-chain degradation; the metabolites have been well identified and measured. The levels of these metabolites, carboxyethyl hydroxychroman (CEHC) and carboxymethylbutyl hydroxychroman (CMBHC), in blood and tissues can be higher than d-T3. Because d-T3 and its metabolites have direct contact with colon epithelial cells, the cells can take up the compound directly without going through the systemic route. Of note is that d-T3 produces the same metabolites as d-T. In the HPLC analysis, all forms of tocotrienols can be efficiently analyzed together with all forms of tocopherols and their metabolites. Therefore, the analysis of d-T3 in colon tumors and normal tissues can give an overall profile of d-T3 and its metabolites and an overall profile of vitamin E nutrition and metabolism in the colon as well as in colon neoplasia.

In one aspect, disclosed herein are methods of reducing, inhibiting, decreasing, and/or preventing the progression of a neoplasm to a cancer (including, but not limited to lung cancer, ovarian cancer, breast cancer, rectal cancer, pancreatic, colon cancer, or intraductal papillary mucinous neoplasm (IPMN) to pancreatic ductal adenocarcinoma) in a subject comprising administering to a subject a therapeutically effective amount of a composition comprising δ-tocotrienol (d-T3) (such as for example, a composition comprising a dose of d-T3 between about 400 mg and 800 mg). In some aspects, the cancer progression being inhibiting is progression occurring following surgical removal or anti-cancer treatment of a cancer.

The disclosed compositions and methods can be used to treat any disease where uncontrolled cellular proliferation occurs such as cancers. A non-limiting list of different types of cancers is as follows: lymphomas (Hodgkins and non-Hodgkins), leukemias, carcinomas, carcinomas of solid tissues, squamous cell carcinomas, adenocarcinomas, sarcomas, gliomas, high grade gliomas, blastomas, neuroblastomas, plasmacytomas, histiocytomas, melanomas, adenomas, hypoxic tumours, myelomas, AIDS-related lymphomas or sarcomas, metastatic cancers, or cancers in general.

A representative but non-limiting list of cancers that the disclosed compositions can be used to treat is the following: lymphomas such as B cell lymphoma and T cell lymphoma; mycosis fungoides; Hodgkin's Disease; myeloid leukemia (including, but not limited to acute myeloid leukemia (AML) and/or chronic myeloid leukemia (CML)); bladder cancer; brain cancer; nervous system cancer; head and neck cancer; squamous cell carcinoma of head and neck; renal cancer; lung cancers such as small cell lung cancer, non-small cell lung carcinoma (NSCLC), lung squamous cell carcinoma (LUSC), and Lung Adenocarcinomas (LUAD); neuroblastoma/glioblastoma; ovarian cancer; pancreatic cancer; prostate cancer; skin cancer; hepatic cancer; melanoma; squamous cell carcinomas of the mouth, throat, larynx, and lung; cervical cancer; cervical carcinoma; breast cancer including, but not limited to triple negative breast cancer; genitourinary cancer; pulmonary cancer; esophageal carcinoma; head and neck carcinoma; large bowel cancer; hematopoietic cancers; testicular cancer; and colon and rectal cancers.

In one aspect the d-T3 composition can be greater than 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100% d-T3. In some aspects the d-T3 can be comprised of D-amino acids or L-amino acids or other synthetic variants of d-T3.

As described above, the δ-tocotrienol comprising compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The δ-tocotrienol comprising compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The δ-tocotrienol comprising compositions may be administered orally, parenterally (e.g., intravenously), by intramuscular injection, by intraperitoneal injection, transdermally, extracorporeally, topically or the like, including topical intranasal administration or administration by inhalant.

As used herein, “topical intranasal administration” means delivery of the δ-tocotrienol comprising compositions into the nose and nasal passages through one or both of the nares and can comprise delivery by a spraying mechanism or droplet mechanism, or through aerosolization of the nucleic acid or vector. Administration of the δ-tocotrienol comprising compositions by inhalant can be through the nose or mouth via delivery by a spraying or droplet mechanism. Delivery can also be directly to any area of the respiratory system (e.g., lungs) via intubation. The exact amount of the δ-tocotrienol comprising compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

δ-tocotrienol comprising compositions for oral administration include powders or granules, suspensions or solutions in water or non-aqueous media, capsules, sachets, or tablets. Thickeners, flavorings, diluents, emulsifiers, dispersing aids or binders may be desirable.

Some of the δ-tocotrienol comprising compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

Effective dosages and schedules for administering the δ-tocotrienol comprising compositions may be determined empirically, and making such determinations is within the skill in the art. The dosage ranges for the administration of the δ-tocotrienol comprising compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above. In one aspect, the daily dose of d-T3 comprising composition can be between about 5 μg and 1000 mg, preferably between about 5 and 1000 mg, more preferably between about 100 and 800 mg, most preferably between about 400 and 800 mg. For example, the daily dose of d-T3 can be 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950 μg, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500, 550, 600, 650, 700, 750, 800, 850, 900, 950, 1000, 1200, 1400, 1600, 1800, 2000, 2200, 2400, 2600, 2800, 3000, or 3200 mg. It is understood and herein contemplated that the effective dose can also be expressed in molar concentration. In one aspect, the effective daily dose of d-T3 can comprise 5, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400 μM.

In one aspect, it is understood and herein contemplated that the d-T3 composition can be administered as a single dose or multiple times in a single day to achieve the daily dosage. In one aspect, disclosed herein are methods of inhibiting, decreasing, reducing, and/or preventing progression of a neoplasm (such as for example, IPMN) to a cancer (such as, for example, recurrence of lung, colon, rectal, ovarian, pancreatic, and/or breast cancer) comprising administering to the subject a therapeutically effective amount of a composition comprising δ-tocotrienol (d-T3), wherein the d-T3 composition is administered 1, 2, 3, 4, 5, 6, 7, 8, 9, 10, 11, 12, 13, 14, 15, 16, 17, 18, 19, 20, 21, 22, 23, 24 times per day.

It is further understood and herein contemplated that the compositions comprising d-T3 can be formulated to have prolonged release of d-T3 or the effective dosage can be administered less frequently than daily administration. Accordingly, disclosed herein are methods of inhibiting, decreasing, reducing, and/or preventing progression of a neoplasm (such as for example, IPMN) to a cancer (such as, for example, recurrence of lung, colon, rectal, ovarian, pancreatic, and/or breast cancer) comprising administering to the subject a therapeutically effective amount of a composition comprising δ-tocotrienol (d-T3), wherein the d-T3 composition is administered one time every 2, 3, 4, 6, 8, 12, 18, 24, 36, 48, 60, 72 hours, 4, 5, 6, 7, 10, 14 days, 3, 4, 5, 6, 7, 8 weeks, 3, 4, 5, 6, 7, 8, 9, 10, 11, 12 months.

While a single administration of d-T3 for the further prevention of cancer recurrence, first occurrence, metastasis, and/or post-treatment maintenance would be ideal, it is understood and herein contemplated that to inhibit cancer recurrence, first occurrence, metastasis, and/or post-treatment maintenance the disclosed d-T3 composition may need to be administered for an extended period of time or the remaining life of the subject. Thus, in one aspect, disclosed herein are methods of inhibiting, decreasing, reducing, and/or preventing progression of a neoplasm (such as for example, IPMN) to a cancer (such as, for example, recurrence of lung, colon, rectal, ovarian, pancreatic, and/or breast cancer) comprising administering to the subject a therapeutically effective amount of a composition comprising δ-tocotrienol (d-T3), wherein the d-t3 composition is administered for at least 1, 2, 3, 4, 5, 6, 7, 8, 9, 10, 11, 12, 13, 14 days, 3, 4, 5, 6, 7, 8 weeks, 3, 4, 5, 6, 7, 8, 9, 10, 11, 12, 18 months, 2, 3, 4, 5, 6, 7, 8, 9, 10, or more years.

The Use of Aspirin in Combination with d-T3 for CRC Prevention:

The landmark endorsement by the US Preventive Services Task Force of aspirin for primary prevention of CRC in 2016 implies that, from now on, aspirin can be the backbone of any novel strategy to prevent CRC. The data clearly demonstrated that d-T3 augments aspirin activity in several cell and animal models of CRC. The interaction between aspirin and d-T3 is an intriguing scientific issue that can be investigated in this project. Both d-T3/metabolites and aspirin have been proposed to inhibit COX-1/2. This only produces an additive effect for the two agents. The enhanced β-catenin degradation, in combination with the additive effect in the inhibition of COX-1/2, can produce synergy. It is well established that the initiation of CRC predominantly involves activation of the Wnt pathway, mostly due to APC mutation, resulting in the accumulation in β-catenin in the nucleus. Another important driving force is the promotion of tumorigenesis by inflammation, which involves activation of the redox-sensitive transcription factor NF-κB and COX-2. The suppression of NF-κB by the antioxidant property of d-T3 (or the inhibition of 15-LOX or alteration of sphingolipid metabolism by d-T3), in combination with the additive effect in the inhibition of COX-1/2, can also produce synergistic effects. Accordingly, disclosed herein are methods of inhibiting, decreasing, reducing, and/or preventing progression of a neoplasm (such as for example, IPMN) to a cancer (such as, for example, recurrence of lung, colon, rectal, ovarian, pancreatic, and/or breast cancer) comprising administering to the subject a therapeutically effective amount of a composition comprising δ-tocotrienol (d-T3), further comprising administering to the subject aspirin.

The usage of aspirin and appropriate dosage can be determined empirically for the subject by a physician. Nevertheless, disclosed herein are methods of inhibiting, decreasing, reducing, and/or preventing progression of a neoplasm (such as for example, IPMN) to a cancer (such as, for example, recurrence of lung, colon, rectal, ovarian, pancreatic, and/or breast cancer) comprising administering to the subject a therapeutically effective amount of a composition comprising δ-tocotrienol (d-T3) and aspirin, wherein the aspirin daily dosage is between about 50 and 1000 mg, preferably between about 50 and 500 mg, more preferably between about 50 and 325 mg. For example, disclosed herein are methods wherein the effective daily dosage of aspirin comprises 50, 55, 60, 65, 70, 70, 71, 72, 73, 74, 75, 76, 77, 78, 79, 80, 81, 82, 83, 84, 85, 90, 95, 100, 125, 150, 175, 200, 225, 250, 275, 300, 325, 350, 375, 400, 425, 450, 475, 500, 550, 600, 650, 700, 750, 800, 850, 900, 950, or 1000 mg. It is understood and herein contemplated that the use of d-T3 in combination with aspirin lowers the effective dosage (i.e., increases the efficacy) of aspirin.

### 1. Antioxidants

The disclosed compositions can comprise antioxidants. Generally, antioxidants are compounds that get react with, and typically get consumed by, oxygen. Since antioxidants typically react with oxygen, antioxidants also typically react with the free radical generators, and free radicals. (“The Antioxidants—The Nutrients that Guard Your Body” by Richard A. Passwater, Ph. D., 1985, Keats Publishing Inc., which is herein incorporated by reference at least for material related to antioxidants). The compositions can contain any antioxidants, and a non-limiting list would including, but not be limited to, non-flavonoid antioxidants and nutrients that can directly scavenge free radicals including multi-carotenes, beta-carotenes, alpha-carotenes, gamma-carotenes, lycopene, lutein and zeanthins, selenium, Vitamin E, including alpha-, beta- and gamma-(tocopherol, particularly .alpha.-tocopherol, etc., vitamin E succinate, and trolox (a soluble Vitamin E analog) Vitamin C (ascorbic acid) and Niacin (Vitamin B3, nicotinic acid and nicotinamide), Vitamin A, 13-cis retinoic acid, N-acetyl-L-cysteine (NAC), sodium ascorbate, pyrrolidin-edithio-carbamate, and coenzyme Q10; enzymes which catalyze the destruction of free radicals including peroxidases such as glutathione peroxidase (GSHPX) which acts on H₂O₂ and such as organic peroxides, including catalase (CAT) which acts on H₂O₂, superoxide dismutase (SOD) which disproportionates O₂H₂O₂; glutathione transferase (GSHTx), glutathione reductase (GR), glucose 6-phosphate dehydrogenase (G6PD), and mimetics, analogs and polymers thereof (analogs and polymers of antioxidant enzymes, such as SOD, are described in, for example, U.S. Pat. No. 5,171,680 which is incorporated herein by reference for material at least related to antioxidants and antioxidant enzymes); glutathione; ceruloplasmin; cysteine, and cysteamine (beta-mercaptoethylamine) and flavenoids and flavenoid like molecules like folic acid and folate. A review of antioxidant enzymes and mimetics thereof and antioxidant nutrients can be found in Kumar et al, Pharmac. Ther. Vol 39:301, 1988 and Machlin L. J. and Bendich, F.A.S.E.B. Journal Vol. 1:441-445, 1987 which are incorporated herein by reference for material related to antioxidants.

### 2. Pharmaceutical Carriers/Delivery of Pharmaceutical Products

As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, including topical intranasal administration or administration by inhalant. As used herein, “topical intranasal administration” means delivery of the compositions into the nose and nasal passages through one or both of the nares and can comprise delivery by a spraying mechanism or droplet mechanism, or through aerosolization of the nucleic acid or vector. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and Mckenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

### a) Pharmaceutically Acceptable Carriers

The compositions, including antibodies, can be used therapeutically in combination with a pharmaceutically acceptable carrier.

Suitable carriers and their formulations are described in Remington: The Science and Practice of Pharmacy (19th ed.) ed. A. R. Gennaro, Mack Publishing Company, Easton, PA 1995. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, antiinflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed antibodies can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

### b) Therapeutic Uses

Effective dosages and schedules for administering the compositions may be determined empirically, and making such determinations is within the skill in the art. The dosage ranges for the administration of the compositions are those large enough to produce the desired effect in which the symptoms of the disorder are effected. The dosage should not be so large as to cause adverse side effects, such as unwanted cross-reactions, anaphylactic reactions, and the like. Generally, the dosage will vary with the age, condition, sex and extent of the disease in the patient, route of administration, or whether other drugs are included in the regimen, and can be determined by one of skill in the art. The dosage can be adjusted by the individual physician in the event of any counterindications. Dosage can vary, and can be administered in one or more dose administrations daily, for one or several days. Guidance can be found in the literature for appropriate dosages for given classes of pharmaceutical products. For example, guidance in selecting appropriate doses for antibodies can be found in the literature on therapeutic uses of antibodies, e.g., Handbook of Monoclonal Antibodies, Ferrone et al., eds., Noges Publications, Park Ridge, N.J., (1985) ch. 22 and pp. 303-357; Smith et al., Antibodies in Human Diagnosis and Therapy, Haber et al., eds., Raven Press, New York (1977) pp. 365-389. A typical daily dosage of the antibody used alone might range from about 1 μg/kg to up to 100 mg/kg of body weight or more per day, depending on the factors mentioned above.

It is understood and herein contemplated that the disclosed methods of inhibiting, reducing, decreasing, and/or preventing the progression of a neoplasm to a cancer (such as, for example, the progression of intraductal papillary mucinous neoplasm (IPMN) to pancreatic ductal adenocarcinoma) comprising administering to the subject a therapeutically effective amount of a composition comprising δ-tocotrienol (d-T3) can further comprise the continued administration of an anti-cancer agent. The anti-tumor agent can comprise any anti-tumor agent known in the art including, but not limited to Abemaciclib, Abiraterone Acetate, Abitrexate (Methotrexate), Abraxane (Paclitaxel Albumin-stabilized Nanoparticle Formulation), ABVD, ABVE, ABVE-PC, AC, AC-T, Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alunbrig (Brigatinib), Ambochlorin (Chlorambucil), Amboclorin Chlorambucil), Amifostine, Aminolevulinic Acid, Anastrozole, Aprepitant, Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Atezolizumab, Avastin (Bevacizumab), Avelumab, Axitinib, Azacitidine, Bavencio (Avelumab), BEACOPP, Becenum (Carmustine), Beleodaq (Belinostat), Belinostat, Bendamustine Hydrochloride, BEP, Besponsa (Inotuzumab Ozogamicin), Bevacizumab, Bexarotene, Bexxar (Tositumomab and Iodine I 131 Tositumomab), Bicalutamide, BiCNU (Carmustine), Bleomycin, Blinatumomab, Blincyto (Blinatumomab), Bortezomib, Bosulif (Bosutinib), Bosutinib, Brentuximab Vedotin, Brigatinib, BuMel, Busulfan, Busulfex (Busulfan), Cabazitaxel, Cabometyx (Cabozantinib-S-Malate), Cabozantinib-S-Malate, CAF, Campath (Alemtuzumab), Camptosar, (Irinotecan Hydrochloride), Capecitabine, CAPOX, Carac (Fluorouracil-Topical), Carboplatin, CARBOPLATIN-TAXOL, Carfilzomib, Carmubris (Carmustine), Carmustine, Carmustine Implant, Casodex (Bicalutamide), CEM, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clafen (Cyclophosphamide), Clofarabine, Clofarex (Clofarabine), Clolar (Clofarabine), CMF, Cobimetinib, Cometriq (Cabozantinib-S-Malate), Copanlisib Hydrochloride, COPDAC, COPP, COPP-ABV, Cosmegen (Dactinomycin), Cotellic (Cobimetinib), Crizotinib, CVP, Cyclophosphamide, Cyfos (Ifosfamide), Cyramza (Ramucirumab), Cytarabine, Cytarabine Liposome, Cytosar-U (Cytarabine), Cytoxan (Cyclophosphamide), Dabrafenib, Dacarbazine, Dacogen (Decitabine), Dactinomycin, Daratumumab, Darzalex (Daratumumab), Dasatinib, Daunorubicin Hydrochloride, Daunorubicin Hydrochloride and Cytarabine Liposome, Decitabine, Defibrotide Sodium, Defitelio (Defibrotide Sodium), Degarelix, Denileukin Diftitox, Denosumab, DepoCyt (Cytarabine Liposome), Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), DTIC-Dome (Dacarbazine), Durvalumab, Efudex (Fluorouracil-Topical), Elitek (Rasburicase), Ellence (Epirubicin Hydrochloride), Elotuzumab, Eloxatin (Oxaliplatin), Eltrombopag Olamine, Emend (Aprepitant), Empliciti (Elotuzumab), Enasidenib Mesylate, Enzalutamide, Epirubicin Hydrochloride, EPOCH, Erbitux (Cetuximab), Eribulin Mesylate, Erivedge (Vismodegib), Erlotinib Hydrochloride, Erwinaze (Asparaginase Erwinia chrysanthemi), Ethyol (Amifostine), Etopophos (Etoposide Phosphate), Etoposide, Etoposide Phosphate, Evacet (Doxorubicin Hydrochloride Liposome), Everolimus, Evista, (Raloxifene Hydrochloride), Evomela (Melphalan Hydrochloride), Exemestane, 5-FU (Fluorouracil Injection), 5-FU (Fluorouracil-Topical), Fareston (Toremifene), Farydak (Panobinostat), Faslodex (Fulvestrant), FEC, Femara (Letrozole), Filgrastim, Fludara (Fludarabine Phosphate), Fludarabine Phosphate, Fluoroplex (Fluorouracil-Topical), Fluorouracil Injection, Fluorouracil-Topical, Flutamide, Folex (Methotrexate), Folex PFS (Methotrexate), FOLFIRI, FOLFIRI-BEVACIZUMAB, FOLFIRI-CETUXIMAB, FOLFIRINOX, FOLFOX, Folotyn (Pralatrexate), FU-LV, Fulvestrant, Gardasil (Recombinant HPV Quadrivalent Vaccine), Gardasil 9 (Recombinant HPV Nonavalent Vaccine), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gleevec (Imatinib Mesylate), Gliadel (Carmustine Implant), Gliadel wafer (Carmustine Implant), Glucarpidase, Goserelin Acetate, Halaven (Eribulin Mesylate), Hemangeol (Propranolol Hydrochloride), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin (Idarubicin Hydrochloride), Idarubicin Hydrochloride, Idelalisib, Idhifa (Enasidenib Mesylate), Ifex (Ifosfamide), Ifosfamide, Ifosfamidum (Ifosfamide), IL-2 (Aldesleukin), Imatinib Mesylate, Imbruvica (Ibrutinib), Imfinzi (Durvalumab), Imiquimod, Imlygic (Talimogene Laherparepvec), Inlyta (Axitinib), Inotuzumab Ozogamicin, Interferon Alfa-2b, Recombinant, Interleukin-2 (Aldesleukin), Intron A (Recombinant Interferon Alfa-2b), Iodine I 131 Tositumomab and Tositumomab, Ipilimumab, Iressa (Gefitinib), Irinotecan Hydrochloride, Irinotecan Hydrochloride Liposome, Istodax (Romidepsin), Ixabepilone, Ixazomib Citrate, Ixempra (Ixabepilone), Jakafi (Ruxolitinib Phosphate), JEB, Jevtana (Cabazitaxel), Kadcyla (Ado-Trastuzumab Emtansine), Keoxifene (Raloxifene Hydrochloride), Kepivance (Palifermin), Keytruda (Pembrolizumab), Kisqali (Ribociclib), Kymriah (Tisagenlecleucel), Kyprolis (Carfilzomib), Lanreotide Acetate, Lapatinib Ditosylate, Lartruvo (Olaratumab), Lenalidomide, Lenvatinib Mesylate, Lenvima (Lenvatinib Mesylate), Letrozole, Leucovorin Calcium, Leukeran (Chlorambucil), Leuprolide Acetate, Leustatin (Cladribine), Levulan (Aminolevulinic Acid), Linfolizin (Chlorambucil), LipoDox (Doxorubicin Hydrochloride Liposome), Lomustine, Lonsurf (Trifluridine and Tipiracil Hydrochloride), Lupron (Leuprolide Acetate), Lupron Depot (Leuprolide Acetate), Lupron Depot-Ped (Leuprolide Acetate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Melphalan, Melphalan Hydrochloride, Mercaptopurine, Mesna, Mesnex (Mesna), Methazolastone (Temozolomide), Methotrexate, Methotrexate LPF (Methotrexate), Methylnaltrexone Bromide, Mexate (Methotrexate), Mexate-AQ (Methotrexate), Midostaurin, Mitomycin C, Mitoxantrone Hydrochloride, Mitozytrex (Mitomycin C), MOPP, Mozobil (Plerixafor), Mustargen (Mechlorethamine Hydrochloride), Mutamycin (Mitomycin C), Myleran (Busulfan), Mylosar (Azacitidine), Mylotarg (Gemtuzumab Ozogamicin), Nanoparticle Paclitaxel (Paclitaxel Albumin-stabilized Nanoparticle Formulation), Navelbine (Vinorelbine Tartrate), Necitumumab, Nelarabine, Neosar (Cyclophosphamide), Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nolvadex (Tamoxifen Citrate), Nplate (Romiplostim), Obinutuzumab, Odomzo (Sonidegib), OEPA, Ofatumumab, OFF, Olaparib, Olaratumab, Omacetaxine Mepesuccinate, Oncaspar (Pegaspargase), Ondansetron Hydrochloride, Onivyde (Irinotecan Hydrochloride Liposome), Ontak (Denileukin Diftitox), Opdivo (Nivolumab), OPPA, Osimertinib, Oxaliplatin, Paclitaxel, Paclitaxel Albumin-stabilized Nanoparticle Formulation, PAD, Palbociclib, Palifermin, Palonosetron Hydrochloride, Palonosetron Hydrochloride and Netupitant, Pamidronate Disodium, Panitumumab, Panobinostat, Paraplat (Carboplatin), Paraplatin (Carboplatin), Pazopanib Hydrochloride, PCV, PEB, Pegaspargase, Pegfilgrastim, Peginterferon Alfa-2b, PEG-Intron (Peginterferon Alfa-2b), Pembrolizumab, Pemetrexed Disodium, Perjeta (Pertuzumab), Pertuzumab, Platinol (Cisplatin), Platinol-AQ (Cisplatin), Plerixafor, Pomalidomide, Pomalyst (Pomalidomide), Ponatinib Hydrochloride, Portrazza (Necitumumab), Pralatrexate, Prednisone, Procarbazine Hydrochloride, Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Radium 223 Dichloride, Raloxifene Hydrochloride, Ramucirumab, Rasburicase, R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Revlimid (Lenalidomide), Rheumatrex (Methotrexate), Ribociclib, R-ICE, Rituxan (Rituximab), Rituxan Hycela (Rituximab and Hyaluronidase Human), Rituximab, Rituximab and, Hyaluronidase Human, Rolapitant Hydrochloride, Romidepsin, Romiplostim, Rubidomycin (Daunorubicin Hydrochloride), Rubraca (Rucaparib Camsylate), Rucaparib Camsylate, Ruxolitinib Phosphate, Rydapt (Midostaurin), Sclerosol Intrapleural Aerosol (Talc), Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sutent (Sunitinib Malate), Sylatron (Peginterferon Alfa-2b), Sylvant (Siltuximab), Synribo (Omacetaxine Mepesuccinate), Tabloid (Thioguanine), TAC, Tafinlar (Dabrafenib), Tagrisso (Osimertinib), Talc, Talimogene Laherparepvec, Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Tecentriq, (Atezolizumab), Temodar (Temozolomide), Temozolomide, Temsirolimus, Thalidomide, Thalomid (Thalidomide), Thioguanine, Thiotepa, Tisagenlecleucel, Tolak (Fluorouracil-Topical), Topotecan Hydrochloride, Toremifene, Torisel (Temsirolimus), Tositumomab and Iodine I 131 Tositumomab, Totect (Dexrazoxane Hydrochloride), TPF, Trabectedin, Trametinib, Trastuzumab, Treanda (Bendamustine Hydrochloride), Trifluridine and Tipiracil Hydrochloride, Trisenox (Arsenic Trioxide), Tykerb (Lapatinib Ditosylate), Unituxin (Dinutuximab), Uridine Triacetate, VAC, Vandetanib, VAMP, Varubi (Rolapitant Hydrochloride), Vectibix (Panitumumab), VeIP, Velban (Vinblastine Sulfate), Velcade (Bortezomib), Velsar (Vinblastine Sulfate), Vemurafenib, Venclexta (Venetoclax), Venetoclax, Verzenio (Abemaciclib), Viadur (Leuprolide Acetate), Vidaza (Azacitidine), Vinblastine Sulfate, Vincasar PFS (Vincristine Sulfate), Vincristine Sulfate, Vincristine Sulfate Liposome, Vinorelbine Tartrate, VIP, Vismodegib, Vistogard (Uridine Triacetate), Voraxaze (Glucarpidase), Vorinostat, Votrient (Pazopanib Hydrochloride), Vyxeos (Daunorubicin Hydrochloride and Cytarabine Liposome), Wellcovorin (Leucovorin Calcium), Xalkori (Crizotinib), Xeloda (Capecitabine), XELIRI, XELOX, Xgeva (Denosumab), Xofigo (Radium 223 Dichloride), Xtandi (Enzalutamide), Yervoy (Ipilimumab), Yondelis (Trabectedin), Zaltrap (Ziv-Aflibercept), Zarxio (Filgrastim), Zejula (Niraparib Tosylate Monohydrate), Zelboraf (Vemurafenib), Zevalin (Ibritumomab Tiuxetan), Zinecard (Dexrazoxane Hydrochloride), Ziv-Aflibercept, Zofran (Ondansetron Hydrochloride), Zoladex (Goserelin Acetate), Zoledronic Acid, Zolinza (Vorinostat), Zometa (Zoledronic Acid), Zydelig (Idelalisib), Zykadia (Ceritinib), and/or Zytiga (Abiraterone Acetate). The treatment methods can include or further include checkpoint inhibitors including, but are not limited to antibodies that block PD-1 (such as, for example, Nivolumab (BMS-936558 or MDX1106), pembrolizumab, CT-011, MK-3475), PD-L1 (such as, for example, atezolizumab, avelumab, durvalumab, MDX-1105 (BMS-936559), MPDL3280A, or MSB0010718C), PD-L2 (such as, for example, rHIgM12B7), CTLA-4 (such as, for example, Ipilimumab (MDX-010), Tremelimumab (CP-675,206)), IDO, B7-H3 (such as, for example, MGA271, MGD009, omburtamab), B7-H4, B7-H3, T cell immunoreceptor with Ig and ITIM domains (TIGIT) (such as, for example BMS-986207, OMP-313M32, MK-7684, AB-154, ASP-8374, MTIG7192A, or PVSRIPO), CD96, B- and T-lymphocyte attenuator (BTLA), V-domain Ig suppressor of T cell activation (VISTA) (such as, for example, JNJ-61610588, CA-170), TIM3 (such as, for example, TSR-022, MBG453, Sym023, INCAGN2390, LY3321367, BMS-986258, SHR-1702, RO7121661), LAG-3 (such as, for example, BMS-986016, LAG525, MK-4280, REGN3767, TSR-033, BI754111, Sym022, FS118, MGD013, and Immutep).

The combination of δ-tocotrienol and anti-cancer agent can be formulated in the same composition of separately. Where separate, the δ-tocotrienol can be administered before, after, or concurrently with the chemotherapeutic agent. Administration of δ-tocotrienol can be administered prophylactically or therapeutically for the inhibition, treatment, reduction, and/or prevention of a cancer or metastasis or prophylactically or therapeutically for the inhibition, treatment, reduction, and/or prevention of a cancer recurrence following therapeutic treatment of a cancer (including resection, radiation, immunotherapy, and/or chemotherapy). It is understood that he use of the δ-tocotrienol provides the advantage of increasing the efficacy of any anti-cancer therapy and thus has the added benefit o flowering dosages of companion therapies and thus can also limit unwanted side effects of those therapies.

### C. Examples

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

Treatment with DT3 is Safe and Induces Apoptosis in the Neoplastic Cells of Patients with IPMN.

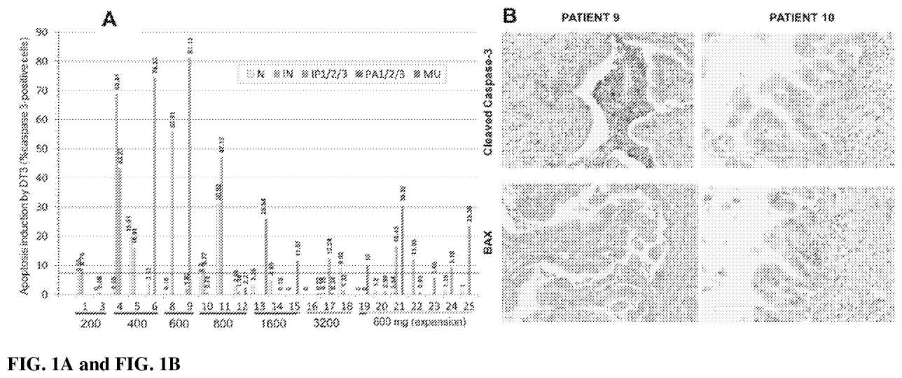

DT3 has been widely used as a dietary supplement for its presumptive benefit in promoting health by decreasing serum cholesterol levels. We have shown that DT3 is well tolerated with no patients experiencing any toxicities in 3 completed phase I trials involving a total of 61 patients treated with doses from 100 to 1600 mg twice daily a day for up to 2 weeks (NCT00985777, NCT01450046, and NCT01446952). In an NCI-sponsored phase 1 study (NCT00985777), we enrolled 9 out of 25 patients with IPMNs who were treated with DT3 for 2 weeks. We found DT3 is well tolerated from 100 to 1600 mg twice daily, with no evidence of toxicity (no dose-limiting toxicities, drug-related adverse events, or changes in the rate of postoperative complications). The half-life of DT3 was approximately 4 hours, with bioavailable serum levels of DT3 with area under the curve (AUC) as high as 150 μM. In our IHC analyses of tumor specimens, we observed a significant induction of cleaved caspase-3 and BAX in the IPMN neoplastic cells of 7 of 9 patients (77%) treated at doses of 100 mg orally twice daily or higher (FIG. 1). We identified 400 mg BID as a Biological Effective Dose (BED) for DT3 in our phase I trial³³. Therefore, we propose to confirm DT3 activity on IPMNs at 400 mg twice daily in a phase II trial, since this was the most bioactive dose observed in our phase I trial.

### MUC4 Expression in IPMNs:

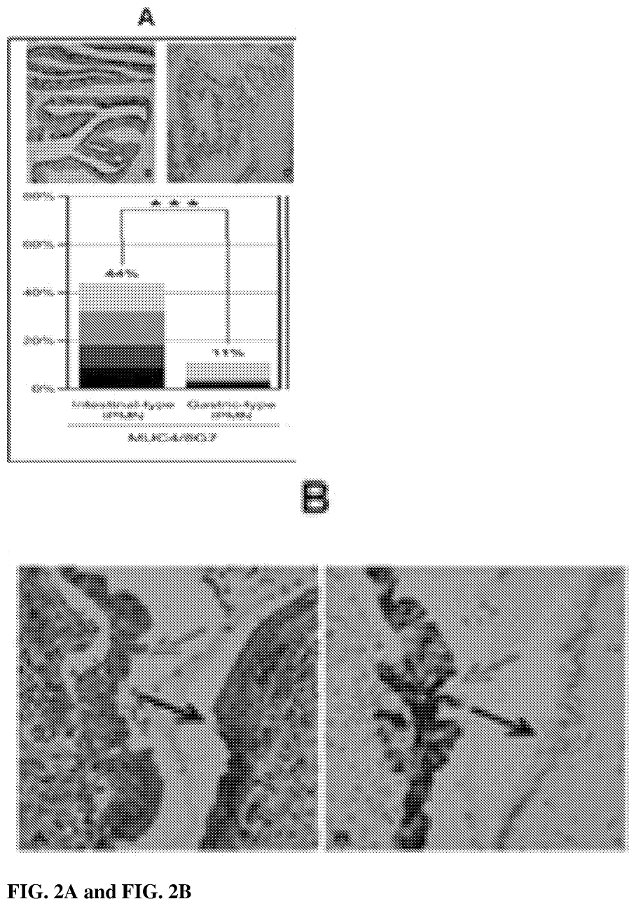

We recently studied the expression profile of MUC4 in 142 IPMNs, including 236 foci of various histological subtypes and dysplastic grades (FIG. 2)¹². Rates of positive expression of MUC4 in intestinal-type IPMNs (considered high risk) were significantly higher than those in the gastric-type IPMNs (considered low risk) (p<. 0001); FIG. 2). The expression of MUC4 increased progressively in the intestinal-type IPMNs. Importantly, even when MUC4 was expressed in gastric-type IPMNs, its expression was truly restricted to cells exhibiting high-grade dysplasia (FIG. 2). Moreover, in a longitudinal follow-up, MUC4 expression was observed during the transition of low-grade lesions to high-grade lesions, and expression was maintained in invasive PC (FIG. 3). Thus, these results suggest that expression of MUC4 in pancreatic lesions is associated with malignant progression.

### Association Between MUC4 Expression and Response to DT3

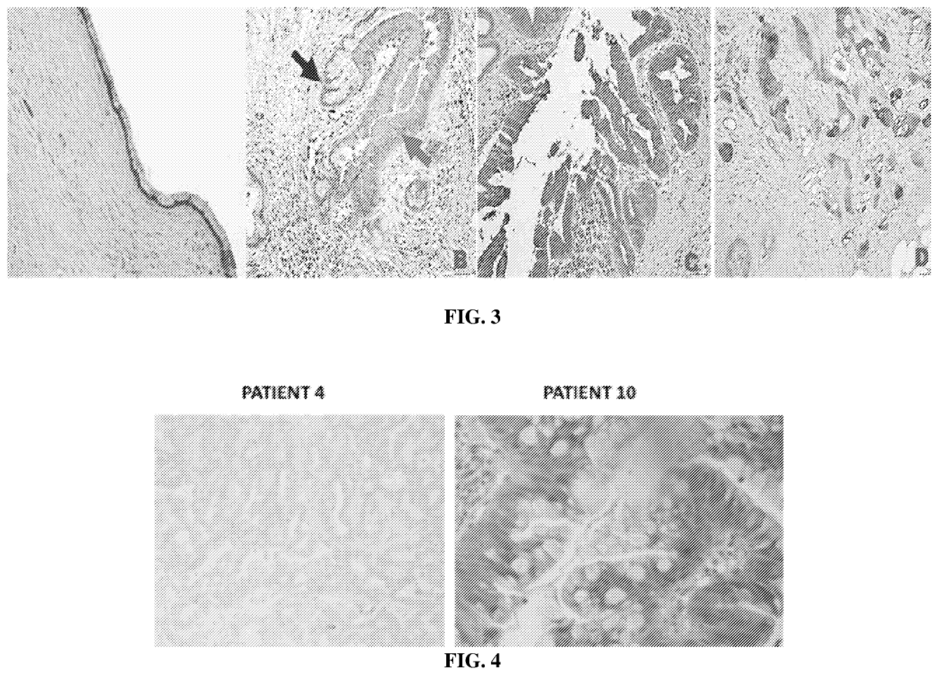

We examined MUC4 expression in patient 4 (responder) and patient 10 (non-responder) (as defined in FIG. 1 above). We observed differential expression of MUC4 in non-responder IPMN cases while no expression of MUC4 was observed in IPMN cases that responded to DT3 (FIG. 4).

Overall, the preliminary studies suggest that: a) DT3 is well tolerated at 400 mg BID dose in patients harboring precursor lesions; b) DT3 induces apoptosis in IPMNs; c) MUC4 expression is associated with high risk IPMNs and increases with their progression to PDA. These studies thus provide a strong rationale presence to undertake Phase II clinical trial to evaluate the ability of DT3 to prevent IPMN progression to PDA (Aim 1). Further, MUC4 is a marker of chemoresistance, as well as malignant IPMNs. Since the analysis of MUC4 in FIG. 4 was performed on resected tissues post-DT3 (lack of pre-treatment samples), it was difficult to determine whether the loss of MUC4 expression in responder is an outcome of therapy response or the of high MUC4 expression is an indicator/predictor of apoptosis-resistant IPMNs. Preliminary studies on a novel MUC4 transgenic model suggest that MUC4 in conjunction with oncogenic Kras drives PDA via IPMN route. Given the significance of MUC4 in PDA pathobiology, it is pertinent to examine the involvement of MUC4 in the context of IPMN progression, and response to DT3.

Currently, there is a scarcity of research that systematically examines agents for chemoprevention of patients with IPMN on AS, underscoring the need to identify and test novel agents in this target population. Based on in vitro, preclinical, and early Phase I trials, DT3 appears to be a promising agent for PDA chemoprevention, establishing the evidence needed to further test this agent in the IPMN AS population. The current randomized, placebo-controlled clinical trial will be the first to evaluate the safety, effectiveness, and mechanism by which DT3 modulates clinical and biological biomarkers implicated in IPMN progression, targeting patients on AS. In addition to biomarkers of clinical progression, we propose to validate several biological markers that have been carefully selected based on our preliminary studies to understand the mechanism by which DT3 modulates IPMN progression. Results from our study will be critical to informing the design of phase 3 clinical trial, ultimately providing a strategy to prevent the progression of IPMN in people on AS, for whom, currently, there are no non-surgical options for reducing the risk of IPMN progression to PDA.

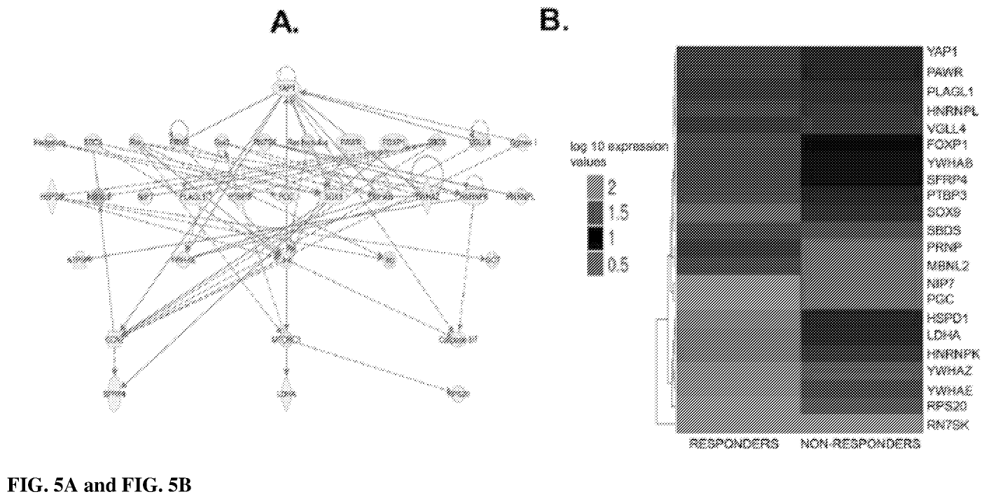

We performed a preliminary RNA seq analysis and compared gene signatures between responders and non-responders to DT3. A careful analysis showed that selective enrichment of pathways associated with apoptosis and cell death. From this analysis, we have found a gene set common to all cell-death and apoptosis-associated pathways (FIG. 5) and their downstream signals converge either at mTORC1 (autophagy, lysosome, nutrient stress, and redox), caspase3/7 (classical apoptosis), or SFRP4 (wnt signaling and apoptosis). Surprisingly, there was no significant difference in pathways associated with redox or hypoxia between responders and non-responders receiving DT3.

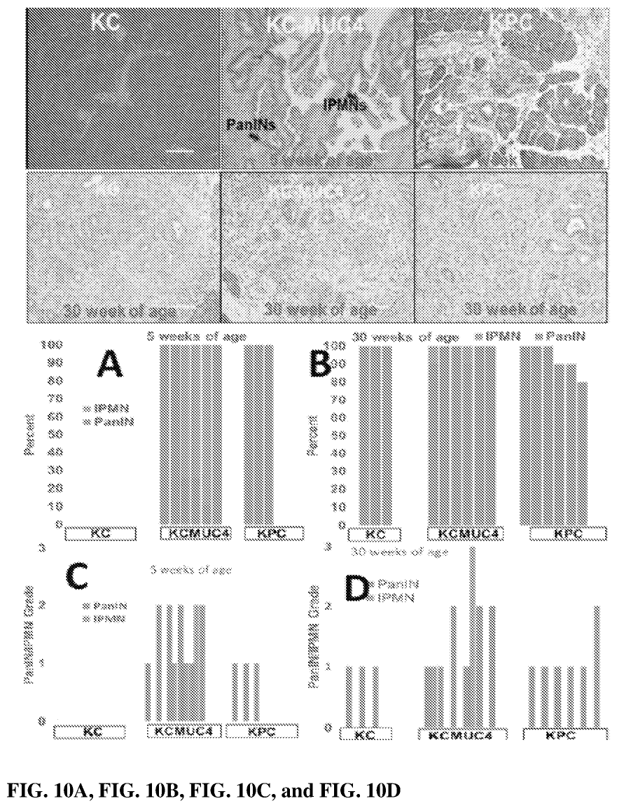

In IPMNs, mutations in KRAS, GNAS, and RNF43 drive tumorigenesis³⁴⁻³⁷, Under the influence of these mutations, premalignant and cancer cells depend on their ability to enforce cell survival pathways and block cell death mechanisms^{34, 69}. Mucin expression occurs universally at the earliest stages of IPMN, with specific mucins associated with specific histological types of IPMNs at various stages of malignant transformation^{38, 39}. More importantly, expression of MUC4 is highly associated with high-risk IPMNs¹² (FIG. 2), suggesting that it is a potential biomarker of IPMNs at risk for malignant progression to PDA. Importantly, our new preliminary data using a novel MUC4 transgenic mouse model suggests that MUC4 in conjunction with KRAS drives pancreatic cancer carcinogenesis via IPMN formation rather than PanIN formation (FIG. 10). Furthermore, recent studies have demonstrated that membrane-bound mucins such as MUC4 impact malignant progression by modifying signal transduction and affecting cell survival through alterations in cell growth, proliferation, death, and autophagy⁷⁰. Recently, we have shown that MUC4 protects pancreatic cancer cells from gemcitabine-induced apoptosis through HER-2/ERK-dependent phosphorylation and inactivation of the proapoptotic protein BAD⁴⁸. This suggests that MUC4 might exert its anti-apoptotic function through ErbB2 downstream signaling. Our preliminary studies suggest that DT3, the most active form of vitamin E compounds not only inhibits the progression of malignant PanIN to PDA but also leads to reduced expression of characteristic biomarkers of malignant IPMN^{10, 28-31}. We have also shown that DT3 antitumor activity is associated with the modulation of multiple downstream targets of oncogenic KRAS, including pERK, pMEK, PAKT, NFKB, p27, p21, BAX, BID, BCL-2, BCLx, and Survivin in models of PDA^{28-31, 33}. Our preliminary data also demonstrate that DT3 targets the Wnt-β-catenin signaling pathway. In this application, we propose studies to assess the effect of DT3 on IPMN oncogenic pathways and programmed cell death. Furthermore, we propose investigating whether mucins and apoptosis proteins can serve as potential biomarkers for IPMN intervention and are modulators of DT3 activity. Lastly, DT3 has an excellent safety profile in humans and induces apoptosis selectively in patient pancreatic tumors, including IPMNs (FIG. 1) 33,45. Taken together, these data support the hypothesis that DT3 will block IPMN progression by targeting pathways that are crucial for IPMN initiation and maintenance.

### DT3 Inhibits Signaling Induced by IPMN Driver Mutations:

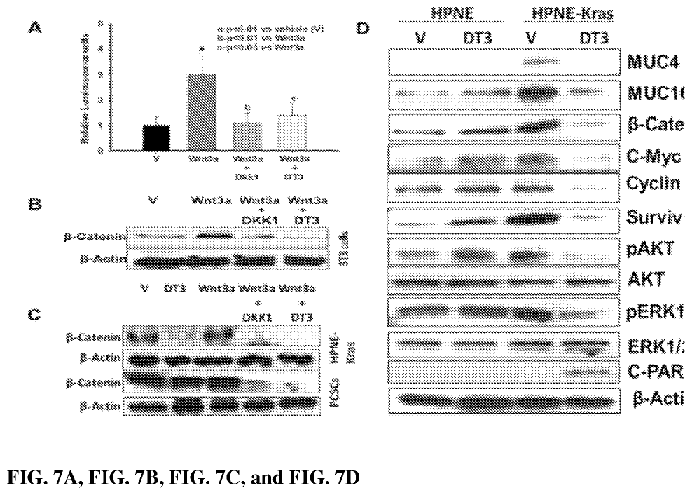

We and others have shown that DT3 inhibits downstream effectors of oncogenic KRAS, such as AKT and ERK, and oncogenic and survival pathways mediated by NF-kB, TRAIL, and c-FLIP. In addition to cell-growth modulation and oncogenic signaling, tocotrienols inhibit several processes important to the growth of micrometastatic cells, such as cancer stem cell-like properties, tumor cell invasion, and angiogenesis. Pancreatic cancer stem cells represent 0.2% to 0.8% of PDA cells and are considered to be responsible for initiating tumor growth, invasion, metastasis, and recurrence. We have also shown that DT3 inhibits PDA stem cell colony formation, invasion, epithelial-mesenchymal transition, growth, and metastasis. Of note, loss of functional mutation in RNF43, a transmembrane E3 ubiquitin ligase that inhibits Wnt/b-catenin signaling by reducing the level of frizzled receptors, is present in 14% to 75% of IPMN cases. However, the impact of these mutations on DT3 bioactivity and their status in murine models that spontaneously form IPMNs, as well as their impact on oncogenic signaling remain obscure. Our preliminary data (FIG. 7), indicates that DT3-induced apoptosis is associated with inhibition of signaling induced by the key IPMN driver mutations (KRAS, GNAS, and RNF43).

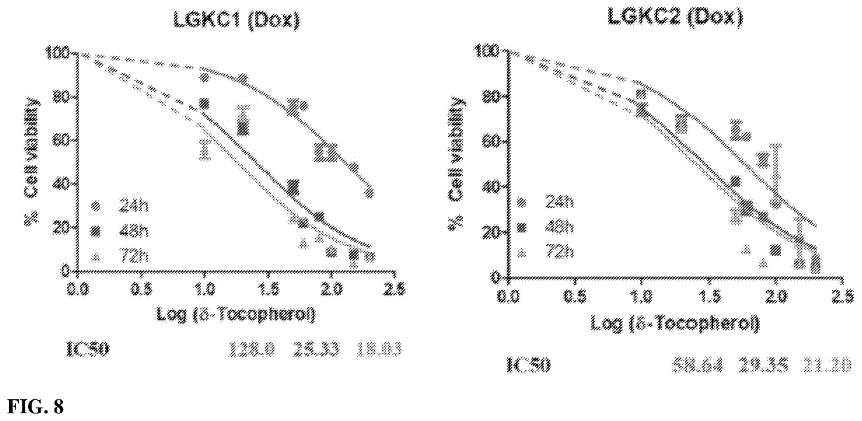

We further studied the effect of DT3 on the proliferation of recently described murine IPMN cell lines LGKC1 & LGKC2 derived from constitutively active Kras and Gnas murine model of pancreatic cystic lesions [p48-Cre; KrasG12D; Rosa26R-rtTA-TetO-GnasR201C]. DT3 suppressed the proliferation of IPMN cell lines in a dose- and time-dependent manner with IC50 of 25.33 μM; 48 h for LGKC1 and 29.35 μM; 48 h for LGKC2 (FIG. 8). These IC50 doses were used for downstream functional studies in LGKC1 and LGKC2 cell lines.

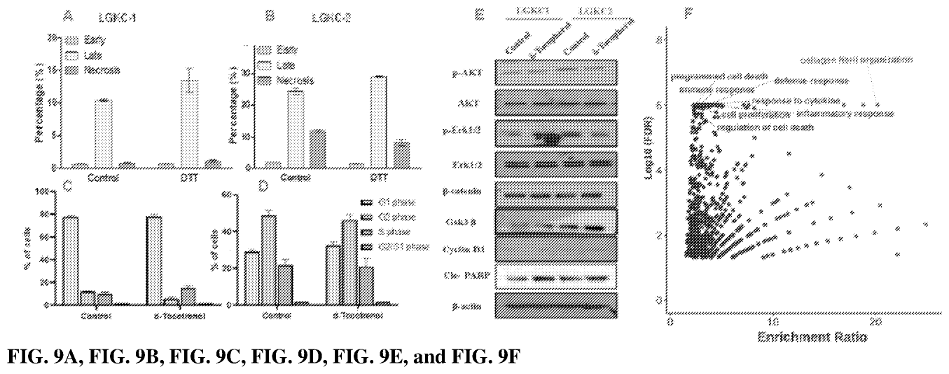

To determine if the reduced cell proliferation of IPMN cells by DT3 were due to its impact on cell cycle or cell death, we assayed apoptosis and cell cycle analysis by flow cytometry. DT3 treatment induced apoptosis in both LGKC1 and LGKC2 o-(FIGS. 9A and 9B). Further, DT3 treatment resulted in G2/M arrest and accumulation of cells in S-phase in LGKC-1 cells while no effect on cell cycle progression was observed in LGKC-2 cells (FIGS. 9C, and 9D). Treatment of IPMN cell lines LGKC1 and LGKC2 (IC50 doses) with DT3 lead to a significant reduction of p-AKT, p-ERK1/2, cyclin D1, upregulation of pGSK3β and cleaved PARP, whereas no effect on expression of b-catenin was observed in both LGKC1 and LGKC2 cell lines (FIG. 9E). RNA seq analysis of the treated cells further indicated significant upregulation of biological processes associated with programmed cell death, cell proliferation, immune response, and collagen fibril organization (FIG. 9F). Overall, these results suggest that DT3 modulates cell death and proliferation signaling induced by IPMN driver mutations.

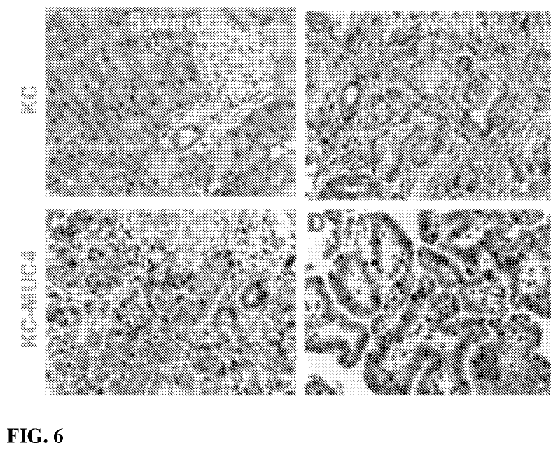

MUC4 transgenic mice develop IPMNs in conjunction with constitutively active Kras: We have recently generated and characterized human MUC4 transgenic (MUC4Tg: tetoMUC4; tTA; PdxCre) that conditionally express human MUC4 in the pancreas. In this Tet-off system, the expression of MUC4 can be switched off by the addition of tetracyclin. The above mice were crossed with LSLKrasG12D mice to generate KC-MUC4 (tetoMUC4; tTA; KrasG12D; PdxCre) mice and compared the progression of precursor lesions with KC and KPC mice (FIG. 10). At 5 weeks of age, KC-MUC4 mice exhibited the appearance of precursor lesions (PanINs and IPMNs), while the pancreata of KC mice appeared normal. The extent of precursor lesions in KC-MUC4 mice was comparable to that observed in KPC mice, suggesting that overexpression of MUC4 accelerates neoplastic transformation induced by mutant Kras. However, the lesions were histologically distinct in KPC and KC-MUC4 mice (FIG. 10). While KPC mice presented purely with PanIN lesions, KC-MUC4 mice exhibited both PanINs and IPMNs, suggesting that MUC4 expression alters the course of progression of precursor lesions. Interestingly, at 30 weeks of age, KC-MUC4 had a comparable proportion of PanINs and invasive PDA to that observed in age-matched KPC; the PanINs were of higher pathological grade. Additionally, KC-MUC4 tumors exhibited increased tumor cell proliferation compared to KC tumors (FIG. 6). We have also developed cell lines from KC-MUC4 and KC mouse models. Cell lines derived from MUC4TG-KC (ABC1631) are morphologically indistinguishable from those derived from KC tumors (ABC1575) and express high levels of human MUC4 in the absence of doxycyclin treatment.

### 1. Example 2

### D. References

Abel E V, Simeone D M. Biology and clinical applications of pancreatic cancer stem cells. Gastroenterology. 2013; 144 (6): 1241-8. PMID: 23622133.
Akahoshi K, Ono H, Akasu M, Ban D, Kudo A, Konta A, Tanaka S, Tanabe M. Rapid growth speed of cysts can predict malignant intraductal mucinous papillary neoplasms. J Surg Res. 2018; 231:195-200. PMID: 30278929.
and prevention efforts for pancreatic cancer: the Florida Pancreas Collaborative. Future Oncol. 2016; 12 (8): 997-1000. PMID: 26863203.
Bafna S, Kaur S, Batra S K. Membrane-bound mucins: the mechanistic basis for alterations in the growth and survival of cancer cells. Oncogene. 2010; 29 (20): 2893-904. PMID: 20348949 PMCID: PMC2879972.
Bafna S, Kaur S, Momi N, Batra S K. Pancreatic cancer cells resistance to gemcitabine: the role of MUC4 mucin. Br J Cancer. 2009; 101 (7): 1155-61. PMID: 19738614 PMCID: PMC2768097.
Batra S K, Kern, H. F., Worlock, A. J., Metzgar, R. S. and Hollingsworth, M. A. Transfection of the human Muc1 mucin gene into a poorly differentiated human pancreatic tumor cell line Panc1: Integration, expression, and ultrastructural changes. J. Cell Science 100, 841-849, 1991. PMID1814933
Benzel J, Fendrich V. Chemoprevention and Treatment of Pancreatic Cancer: Update and Review of the Literature. Digestion. 2018; 97 (4): 275-87. PMID: 29587290.
Boik J C, Newman R A, Boik R J. Quantifying synergism/antagonism using nonlinear mixed-effects modeling: a simulation study. Stat Med. 2008; 27 (7): 1040-61. PMID: 17768754.
Bray F, Ferlay J, Soerjomataram I, Siegel R L, Torre L A, Jemal A. Global cancer statistics 2018: GLOBOCAN estimates of incidence and mortality worldwide for 36 cancers in 185 countries. CA Cancer J Clin. 2018; 68 (6): 394-424. PMID: 30207593.
Carmicheal J, Hayashi C, Huang X, Liu L, Lu Y, Krasnoslobodtsev A, Lushnikov A, Kshirsagar P G, Patel A, Jain M, Lyubchenko Y L, Lu Y, Batra S K*, Kaur S*. Label-free characterization of exosome via surface enhanced Raman spectroscopy for the early detection of pancreatic cancer. Nanomedicine 16:88-96. 2019; PMID 30550805. * both are corresponding authors.
Cauley C E, Waters J A, Dumas R P, Meyer J E, Al-Haddad M A, DeWitt J M, Lillemoe K D, Schmidt C M. Outcomes of primary surveillance for intraductal papillary mucinous neoplasm. J Gastrointest Surg. 2012; 16 (2): 258-67; discussion 66. PMID: 22089952.
Chaturvedi P, Singh A P, Chakraborty S, Chauhan S C, Bafna S, Meza J L, Singh P K, Hollingsworth M A,
Chaturvedi P, Singh A P, Chakraborty S, Chauhan S C, Bafna S., Meza J L, Singh P K., Hollingsworth, M. A., Mehta P P, and Batra S K MUC4 mucin interacts with and stabilizes the HER2 oncoprotein in human pancreatic cancer cells. Cancer Research, 68 (7): 2065-70, 2008 PMID18381409/PMC2835497
Chaturvedi P, Singh A P, Moniaux N, Senapati S, Chakraborty S, Meza J L, Batra S K. MUC4 mucin potentiates pancreatic tumor cell proliferation, survival, and invasive properties and interferes with its interaction to extracellular matrix proteins. Mol Cancer Res. 2007; 5 (4): 309-20. PMID: 17406026.
Chen D T, Davis-Yadley A H, Huang P Y, Husanin K, Centeno B A. Permuth-Wey, Pimiento J M, Malafa M. Prognostic Fifteen-Gene Signature for Ealry Stage Pancreatic Ductal Adenocarcinoma. PLos Onw. 2015 Aug. 6; 10 (8): e0133562. Doi: 10.1371/journal.pone.0133562. eCollection 2015. PMCID: PMC4527782
Chou T C, Talalay P. Quantitative analysis of dose-effect relationships: the combined effects of multiple drugs or enzyme inhibitors. Adv Enzyme Regul. 1984; 22:27-55. PMID: 6382953.
Chugh S, Barkeer S, Rachagani S, Nimmakayala R K, Perumal N, Pothuraju R, Atri P, Mahapatra S, Thapa I, Talmon G A, Smith L M, Yu X, Neelamegham S, Fu J, Xia L, Ponnusamy M P, and Batra S K. Disruption of Clgaltl Gene Promotes Development and Metastasis of Pancreatic Adenocarcinomas in Mice. Gastroenterology 155:1608-1624, 2018. PMID: 30086262
Costa-Silva B, Aiello N M, Singh S, Zhang H, Thakur B K, Becker A, Hoshino A, Mark M T, Molina H, Xiang J, Zhang T, Theilen T M, García-Santos G, Williams C, Ararso Y, Huang Y, Rodrigues G, Shen T L, Labori K J, Lothe I M B, Kure E H, Ocean A J, Hernandez J, Doussot A, Ebbesen S, Grandgenett P, Hollingsworth M A, Jain M, Mallya K, Batra S K, Jarnagin W R, Schwartz R E, Matei I, Peinado H, Stanger B, Bromberg J, and Lyden D V (2015). Pancreatic cancer exosomes initiate pre-metastatic niche formation in the liver. Nature Cell Biology 17 (6): 816-26, 2015. PMID: 25985394
D'Angelica M I, Jarnagin W R, Do R K, Brennan M F, Allen P J. Malignant progression in IPMN: a cohort analysis of patients initially selected for resection or observation. Ann Surg Oncol. 2013; 20 (2): 440-7. PMID: 23111706.
Das K K, Mullady D K. Growth in Branch Duct IPMN Undergoing Surveillance: Unsafe at Any Speed? Clin Gastroenterol Hepatol. 2018; 16 (9): 1401-3. PMID: 29775796.
Davis-Yadley A H, Malafa M P. Vitamins in pancreatic cancer: a review of underlying mechanisms and future applications. Adv Nutr. 2015; 6 (6): 774-802. PMID: 26567201 PMCID: PMC4642423.
Del Chiaro M, Ateeb Z, Hansson M R, Rangelova E, Segersvard R, Kartalis N, Ansorge C, Lohr M J, Arnelo U, Verbeke C. Survival Analysis and Risk for Progression of Intraductal Papillary Mucinous Neoplasia of the Pancreas (IPMN) Under Surveillance: A Single-Institution Experience. Ann Surg Oncol. 2017; 24 (4): 1120-6. PMID: 27822633 PMCID: PMC5339331.
different forms of tocopherols and tocotrienols. Mol Carcinog. 2020; 59 (4): 365-89. PMID: 32017273.
European Study Group on Cystic Tumours of the P. European evidence-based guidelines on pancreatic cystic neoplasms. Gut. 2018; 67 (5): 789-804. PMID: 29574408 PMCID: PMC5890653.
Fonseca A L, Kirkwood K, Kim M P, Maitra A, Koay E J. Intraductal Papillary Mucinous Neoplasms of the Pancreas: Current Understanding and Future Directions for Stratification of Malignancy Risk. Pancreas. 2018; 47 (3): 272-9. PMID: 29424809 PMCID: PMC5808987.
for Chemoprevention of Pancreatic Cancer. Curr Med Chem. 2018; 25 (22): 2535-44. PMID: 28183260.
Francois R A, Zhang A, Husain K, Wang C, Hutchinson S, Kongnyuy M, Batra S K, Coppola D, Sebti S M, Malafa M P. Vitamin E delta-tocotrienol sensitizes human pancreatic cancer cells to TRAIL-induced apoptosis through proteasome-mediated down-regulation of c-FLIPs. Cancer Cell Int. 2019; 19:189. PMID: 31367187 PMCID: PMC6647259.
Francois R A, Zhang A, Husain K, Wang C, Hutchinson S, Kongnyuy M, Batra S K, Coppola D, Sebti S M, Malafa M P. Vitamin E delta-tocotrienol sensitizes human pancreatic cancer cells to TRAIL-induced apoptosis through proteasome-mediated down-regulation of c-FLIPs. Cancer Cell Int. 2019; 19:189. PMID: 31367187 PMCID: PMC6647259.
G, Richmond E, Rodriguez L M, Bigg M, Schnoll-Sussmans F, Meyskens F. Phase IIA trial testing erlotinib as an intervention against intraductal pancreatic mucinous neoplasms. Cancer Prev Res (Phila). 2011; 4 (4): 512-3. PMID: 21464031 PMCID: PMC4492452.
Gangi A, Malafa M, Klapman J. Endoscopic Ultrasound-Based Pancreatic Cancer Screening of High-Risk Individuals: A Prospective Observational Trial. Pancreas. 2018 May/Jun; 47 (5): 586-591. doi: 10.1097/MPA.0000000000001038. PMID: 29683970
Ganguly K, Krishn S R, Rachagani S, Jahan R, Shah A, Nallasamy P, Rauth S, Atri P, Cox J L, Pothuraju R, Smith L M, Ayala S, Evans C, Ponusamy M P, Kumar S, Kaur S, Batra S K. Secretory mucin 5AC promotes neoplastic progression by augmenting KLF4-mediated pancreatic cancer cell stemness. Cancer Research 81 (1), 91-102, 2021. PMID33127746
Gausman V, Kandel P, Van Riet P A, Moris M, Kayal M, Do C, Poneros J M, Sethi A, Gress F G, Schrope B A, Luk L, Hecht E, Jovani M, Bruno M J, Cahen D L, Wallace M B, Gonda T A. Predictors of Progression Among Low-Risk Intraductal Papillary Mucinous Neoplasms in a Multicenter Surveillance Cohort. Pancreas. 2018; 47 (4): 471-6. PMID: 29521942.
Gautam S K, Kumar S, Dam V, Ghersi D, Jain M, and Batra S K. MUCIN-4 (MUC4) is a novel tumor antigen in pancreatic cancer immunotherapy. Seminars in Immunol. 47:101391, 2020. PMID31952903
Gautam S K, Kumar S, Dam V, Ghersi D, Jain M, Batra S K. MUCIN-4 (MUC4) is a novel tumor antigen in pancreatic cancer immunotherapy. Semin Immunol. 2020:101391. PMID: 31952903.
Gordon Lan K K, DeMets D L. Discrete sequential boundaries for clinical trials. Biometrika.
Hayashi T, Ishiwatari H, Ihara H, Kawano Y, Takada K, Miyanishi K, Kobune M, Takimoto R, Sonoda T, Takayama T, Kato J, Niitsu Y. Suppressive effect of sulindac on branch duct-intraductal papillary mucinous neoplasms. J Gastroenterol. 2009; 44 (9): 964-75. PMID: 19536452.
Hodul P J, Dong Y, Husain K, Pimiento J M, Chen J, Zhang A, Francois R, Pledger W J, Coppola D, Sebti S M, Chen D T, Malafa M P. Vitamin E delta-tocotrienol induces p27 (Kip1)-dependent cell-cycle arrest in pancreatic cancer cells via an E2F-1-dependent mechanism. PLOS One. 2013; 8 (2): e52526. PMID: 23393547 PMCID: PMC3564846.
Hodul P J, Dong Y, Husain K, Pimiento J M, Chen J, Zhang A, Francois R, Pledger W J, Coppola D, Sebti S M, Chen D T, Malafa M P. Vitamin E delta-tocotrienol induces p27 (Kip1)-dependent cell-cycle arrest in pancreatic cancer cells via an E2F-1-dependent mechanism. PLOS One. 2013; 8 (2): e52526. PMID: 23393547 PMCID: PMC3564846.
holy grail for the early detection of pancreatic cancer. Future Oncol. 2015; 11 (23): 3105-8. PMID: 26549701 PMCID: PMC7109591.
Hoshino A, Costa-Silva B, Shen T L, Rodrigues G, Hashimoto A, Mark M T, Molina H, Kosaka S, Giannatale A D, Ceder S, Singh S, Williams C, Soplop N, Uryu K, Pharmer L, King T, Bojmar L, Davies A E, Ararso Y. Zhang T, Zhang H, Hernandez J, Weiss J M, Dumont-Cole V D, Kramer K, Wexler L H, Narendran A, Schwartz G, Healey J H, Sandstrom P, Labori K J, Kure E H, Grandgenett P M, Hollingsworth M A, de Sousa M, Kaur S, Jain M, Mallya K, Batra S K, Jarnagin W R, Brady M S, Fodstad O, Muller V, Pantel K, Minn A J, Bissell M J, Garcia B A, Kang T, Rajesekhar V, Ghajar C M, Matei I Peinado H, Bromberg J and Lyden D. Tumour exosome integrins determine organotropic metastasis. Nature, 527 (7578): 329-35, 2015: PMID 26524530
Husain K, Centeno B A, Chen D T, Fulp W J, Perez M, Zhang Lee G, Luetteke N, Hingorani S R, Sebti S M, and Malafa M P. Prolonged survival and delayed progression of pancreatic intraepithelial neoplasia in LSL-KrasG12D/+; Pdx-1Cre mice by vitamin E o-tocotrienol. Carcinogenesis. 2013 Jan. 23.
Husain K, Centeno B A, Chen D T, Fulp W J, Perez M, Zhang Lee G, Luetteke N, Hingorani S R, Sebti S M,
Husain K, Centeno B A, Chen D T, Hingorani S R, Sebti S M, and Malafa M P. Vitamin E Delta-tocotrienol prolongs survival in the LSL-KrasG12D/+: LSL-Trp53R172H/+: Pdx-1-Cre (KPC) transgenic mouse model of pancreatic cancer. Cancer Prev Res (Phila). 2013 October; 6 (10): 1074-83. doi: 10.1158/1940-6207.CAPR-13-0157. Epub 2013 Aug. 20. PMCID: PMC4165552.
Husain K, Centeno B A, Chen D T, Hingorani S R, Sebti S M, Malafa M P. Vitamin E delta-tocotrienol prolongs survival in the LSL-KrasG12D/+; LSL-Trp53R172H/+; Pdx-1-Cre (KPC) transgenic mouse model of pancreatic cancer. Cancer Prev Res (Phila). 2013; 6 (10): 1074-83. PMID: 23963802 PMCID: PMC4165552.
Husain K, Centeno B A, Coppola D, Trevino J, Sebti S M, Malafa M P. delta-Tocotrienol, a natural form of vitamin E, inhibits pancreatic cancer stem-like cells and prevents pancreatic cancer metastasis. Oncotarget. 2017; 8 (19): 31554-67. PMID: 28404939 PMCID: PMC5458229.
Husain K, Francois R A, Hutchinson S Z, Neuger A M, Lush R, Coppola D, Sebti S, Malafa M P. Vitamin E delta-tocotrienol levels in tumor and pancreatic tissue of mice after oral administration. Pharmacology. 2009; 83 (3): 157-63. PMID: 19142032 PMCID: PMC2834260.
Husain K, Francois R A, Yamauchi T, Perez M, Sebti S M, Malafa M P. Vitamin E delta-tocotrienol augments the antitumor activity of gemcitabine and suppresses constitutive NF-kappaB activation in pancreatic cancer. Mol Cancer Ther. 2011; 10 (12): 2363-72. PMID: 21971120 PMCID: PMC3237822.
Husain K, Francois R A, Yamauchi T, Perez M, Sebti S M, Malafa M P. Vitamin E delta-tocotrienol augments the antitumor activity of gemcitabine and suppresses constitutive NF-kappaB activation in pancreatic cancer. Mol Cancer Ther. 2011; 10 (12): 2363-72. PMID: 21971120 PMCID: PMC3237822.
Husain K, Zhang A, Shivers S, Davis-Yadley A, Coppola D, Yang C S, Malafa M P. Chemoprevention of Azoxymethane-induced Colon Carcinogenesis by Delta-Tocotrienol. Cancer Prev Res (Phila). 2019 June; 12 (6): 357-366. doi: 10.1158/1940-6207.CAPR-18-0290. Epub 2019 Apr. 2. PMID: 30940630
Ibrahim J G, Chu H, Chen L M. Basic concepts and methods for joint models of longitudinal and survival data. J Clin Oncol. 2010; 28 (16): 2796-801. PMID: 20439643 PMCID: PMC4503792.
Ideno N, Yamaguchi H, Ghosh B, Gupta S, Okumura T, Steffen D J, Fisher C G, Wood L D, Singhi A D, Nakamura M, Gutkind J S, Maitra A. GNAS (R201C) Induces Pancreatic Cystic Neoplasms in Mice That Express Activated KRAS by Inhibiting YAPI Signaling. Gastroenterology. 2018; 155 (5): 1593-607 e12. PMID: 30142336 PMCID: PMC6219919.
Ishiwata T, Matsuda Y, Yoshimura H, Sasaki N, Ishiwata S, Ishikawa N, Takubo K, Arai T, Aida J. Pancreatic cancer stem cells: features and detection methods. Pathol Oncol Res. 2018; 24 (4): 797-805. PMID: 29948612.
Jacobsen P B, Shibata D, Siegel E M, Lee J H, Fulp W J, Alemany C, Abesada-Terk G Jr., Brown R, Cartwright T, Faig D, Kim G, Levine R, Markham M J, Schreiber F, Sharp P, and Malafa M P. Evaluating the quality of psychosocial care in outpatient medical oncology settings using performance indicators. Psychooncology. 2011 Nov; 20 (11): 1221-7. PMCID: PMC4497369.
Jahan R, Ganguly K, Smith L M, Atri P, Carmicheal J, Sheinin Y, Rachagani S, Natarajan G, Brand R E, Macha MA, Grandgenett P M, Kaur S*, Batra S K. Trefoil factor(s) and CA19.9: A promising panel for early detection of pancreatic cancer. EBioMedicine. S2352-3964 (19) 30201-4, 2019. PMID 30956167.
Karmakar S, Rauth S, Nallasamy P, Perumal N, Nimmakayala R K, Leon F, Gupta R, Barkeer S, Venkata R C, Raman V, Rachagani S, Ponnusamy M P* and Batra S K*. PAFI Regulates Stem Cell Features of Pancreatic Cancer Cells, Independently of the PAFI Complex, via Interactions with PHFSA and DDX3. Gastroenterology 159 (5), 1898-1915, 2020. * both are corresponding authors. PMID32781084
Kaur S, Sharma N, Krishn S R, Lakshmanan I, Rachagani S, Baine M J, Smith L M, Lele S M, Sasson A R, Guha S, Mallya K, Anderson J M, Hollingsworth M A, Batra S K. MUC4-mediated regulation of acute phase protein lipocalin 2 through HER2/AKT/NF-kappaB signaling in pancreatic cancer. Clin Cancer Res. 2014; 20 (3): 688-700. PMID: 24240113 PMCID: PMC3946494.
Kaur S, Smith L M, Patel A, Melanie M, Watley D C, Malik S S, Krishn S R, Mallya K, Aithal A, Sasson A, Johansson S L, Jain M, Singh S, Guha S, Are C, Raimondo M, Hollingsworth M. A. Brand R E and Batra S K. A Combination of MUC5AC and CA19-9 Improves the Diagnosis of Pancreatic Cancer-A Multicenter Study. American J. Gastroenterology, 112 (1), 172-183, 2017. PMID27845339
Kayal M, Luk L, Hecht E M, Do C, Schrope B A, Chabot J A, Gonda T A. Long-Term Surveillance and
Khannoussi W, Vullierme M P, Rebours V, Maire F, Hentic O, Aubert A, Sauvanet A, Dokmak S, Couvelard A, Hammel P, Ruszniewski P, Levy P. The long term risk of malignancy in patients with branch duct intraductal papillary mucinous neoplasms of the pancreas. Pancreatology. 2012; 12 (3): 198-202. PMID: 22687372.
Kim D, Pertea G, Trapnell C, Pimentel H, Kelley R, Salzberg S L. TopHat2: accurate alignment of transcriptomes in the presence of insertions, deletions and gene fusions. Genome Biol. 2013; 14 (4): R36. PMID: 23618408 PMCID: PMC4053844.
Kitazono I, Higashi M, Kitamoto S, Yokoyama S, Horinouchi M, Osako M, Shimizu T, Tabata M, Batra S K, Goto M, Yonezawa S. Expression of MUC4 mucin is observed mainly in the intestinal type of intraductal papillary mucinous neoplasm of the pancreas. Pancreas. 2013; 42 (7): 1120-8. PMID: 23921963 PMCID: PMC3779537.
Kolb J M, Argiriadi P, Lee K, Liu X, Bagiella E, Gupta S, Lucas A L, Kim M K, Kumta N A, Nagula S, Sarpel U, DiMaio C J. Higher Growth Rate of Branch Duct Intraductal Papillary Mucinous Neoplasms Associates With Worrisome Features. Clin Gastroenterol Hepatol. 2018; 16 (9): 1481-7. PMID: 29535058.
Kumar S, Torres M P, Kaur S, Rachagani S, Joshi S, Johansson S L, Momi N, Baine M J, Gilling C E, Smith L M, Wyatt T A, Jain M, Joshi S S, Batra S K. Smoking accelerates pancreatic cancer progression by promoting differentiation of MDSCs and inducing HB-EGF expression in macrophages. Oncogene 16; 34 (16): 2052-60, 2015. PMID: 24909166
Lafemina J, Katabi N, Klimstra D, Correa-Gallego C, Gaujoux S, Kingham T P, Dematteo R P, Fong Y,
Laffan T A, Horton K M, Klein A P, Berlanstein B, Siegelman S S, Kawamoto S, Johnson P T, Fishman E K, Hruban R H. Prevalence of unsuspected pancreatic cysts on MDCT. AJR Am J Roentgenol. 2008; 191 (3): 802-7. PMID: 18716113 PMCID: PMC2692243.
Lakshmanan I, Seshacharyulu P, Haridas D, Rachagani S, Gupta S, Joshi S, Guda C, Yan Y, Jain M, Ganti A K, Ponnusamy M P, Batra S K. Novel HER3/MUC4 oncogenic signaling aggravates the tumorigenic phenotypes of pancreatic cancer cells. Oncotarget. 2015; 6 (25): 21085-99. PMID: 26035354 PMCID: PMC4673252.
Laronga C, Gray J E, Siegel E M, Lee J H, Fulp W J, Fletcher M, Schreiber F, Brow R, Levine R, Cartwright T. Abesada-Terk G Jr, Kim G, Alemany C, Faig D, Sharp P, Markham M J, Shibata D, Malafa M P, Jacobsen P B. Florida initiative for quality cancer care: improvements in breast cancer quality indicators during a 3-year interval. J Am Coll Surg. 2014 Oct: 29 (4): 638-645.e1, doi: 10.1016/j.jamcollsugr.2014.03.063. Epub 2014 Jul. 1. PMCID: PMC4505727.
Lee J H, Kim Y, Choi J W, Kim Y S. KRAS, GNAS, and RNF43 mutations in intraductal papillary mucinous
Lee K S, Sekhar A, Rofsky N M, Pedrosa I. Prevalence of incidental pancreatic cysts in the adult population on MR imaging. Am J Gastroenterol. 2010; 105 (9): 2079-84. PMID: 20354507.
Lipkin S, Lee J, Imagawa D, Hewitt S M, Tucker C, Zell J A, Wong V, Garcia A, Gonzalez R, Della Zanna
Logsdon C D, Abbruzzese J L. Chemoprevention of pancreatic cancer: ready for the clinic? Cancer Prev Res (Phila). 2010; 3 (11): 1375-8. PMID: 21084259 PMCID: PMC3051830.
Mahipal A, Klapman J, Vignesh S, Yang C S, Neuger A, Chen D T, Malafa M P. Pharmacokinetics and safety of vitamin E delta-tocotrienol after single and multiple doses in healthy subjects with measurement of vitamin E metabolites. Cancer Chemother Pharmacol. 2016; 78 (1): 157-65. PMID: 27278668 PMCID: PMC4939900.
Malafa M P, Corman M M, Shibata D, Siegel E M, Lee J H, and Jacobsen P B. The Florida Initiative for Quality Cancer Care: a regional project to measure and improve cancer care. Cancer Control Journal. 2009 October; 16 (4): 318-27. PMID: 19910918
Malafa M P, Sebti S, inventors, University of South Florida, assignee. Delta-Tocotrienol Treatment and Prevention of Pancreatic Cancer. U.S. Pat. No. 8,288,369 B2 2012 Oct. 16.
Malafa M P. Prolonged survival and delayed progression of pancreatic intraepithelial neoplasia in LSL-KrasG12D/+; Pdx-1-Cre mice by vitamin E delta-tocotrienol. Carcinogenesis. 2013; 34 (4): 858-63. PMID: 23302291 PMCID: PMC3616675.
Marinelli V, Secchettin E, Andrianello S, Moretti C, Donvito S, Marchegiani G, Esposito A, Casetti L, Salvia R. Psychological distress in patients under surveillance for intraductal papillary mucinous neoplasms of the pancreas: The “Sword of Damocles” effect calls for an integrated medical and psychological approach a prospective analysis. Pancreatology. 2020. PMID: 31948794.
Matthaei H, Schulick R D, Hruban R H, Maitra A. Cystic precursors to invasive pancreatic cancer. Nat Rev Gastroenterol Hepatol. 2011; 8 (3): 141-50. PMID: 21383670 PMCID: PMC3236705.
Mehta P P, Batra S K. MUC4 mucin interacts with and stabilizes the HER2 oncoprotein in human pancreatic cancer cells. Cancer Res. 2008; 68 (7): 2065-70. PMID: 18381409 PMCID: PMC2835497.
Miller M S, Allen P, Brentnall T A, Goggins M, Hruban R H, Petersen G M, Rao C V, Whitcomb D C, Brand R E, Chari S T, Klein A P, Lubman D M, Rhim A D, Simeone D M, Wolpin B M, Umar A, Srivastava S, Steele V E, Rinaudo J A. Pancreatic Cancer Chemoprevention Translational Workshop: Meeting Report. Pancreas. 2016; 45 (8): 1080-91. PMID: 27518363 PMCID: PMC4988336.
Mimeault M, Johansson S L, Senapati S, Momi N, Chakraborty S, Batra S K. MUC4 down-regulation
Mohammed A, Janakiram N B, Madka V, Li M, Asch A S, Rao C V. Current Challenges and Opportunities
Mohammed A, Rao C V. Pancreatic Cancer Chemoprevention: Challenges and Opportunities. Curr Med Chem. 2018; 25 (22): 2532-4. PMID: 30014794.
Moniaux N, Junker W M, Singh A P, Jones A M, and Batra S K. Characterization of human mucin MUC17: Complete coding sequence and organization. J Biol Chem. 281 (33): 23676-85, 2006. PMID16737958
Moniaux N, Nemos C, Schmied B, Chauhan S C, Deb S, Morikane K, Choudhury A., Vanlith M, Sutherlin M, Sikela J M., Hollingsworth, M. A. and Batra S K The human homologue of the RNA polymerase II-associated factor 1 (hPaf1), localized on the 19q13 amplicon, is associated with tumorigenesis. Oncogene, 25:3247-57, 2006. PMID: 16491129
Mortazavi A, Williams B A, McCue K, Schaeffer L, Wold B. Mapping and quantifying mammalian transcriptomes by RNA-Seq. Nat Methods. 2008; 5 (7): 621-8. PMID: 18516045.
Moschovis D, Bamias G, Delladetsima I. Mucins in neoplasms of pancreas, ampulla of Vater and biliary system. World J Gastrointest Oncol. 2016; 8 (10): 725-34. PMID: 27795812 PMCID: PMC5064050.
Mukewar S, de Pretis N, Aryal-Khanal A, Ahmed N, Sah R, Enders F, Larson J J, Levy M J, Takahashi N, Topazian M, Pearson R, Vege S S, Chari S T. Fukuoka criteria accurately predict risk for adverseoutcomes during follow-up of pancreatic cysts presumed to be intraductal papillary mucinous neoplasms. Gut. 2017; 66 (10): 1811-
Nagata K, Horinouchi M, Saitou M, Higashi M, Nomoto M, Goto M, Yonezawa S. Mucin expression profile in pancreatic cancer and the precursor lesions. J Hepatobiliary Pancreat Surg. 2007; 14 (3): 243-54. PMID: 17520199.
neoplasm of the pancreas: a meta-analysis. Springerplus. 2016; 5 (1): 1172. PMID: 27512631 PMCID: PMC4960083.
Nimmakayala R K, Leon F, Rachagani S, Rauth S, Nallasamy P, Marimuthu S, Shailendra G K, Chhonker Y S, Chugh S, Chirravuri R, Gupta R, Mallya K, Prajapati D R, Lele S M, C Caffrey T, L Grem J, Grandgenett P M, Hollingsworth M A, Murry D J, Batra S K*, Ponnusamy M P*. Metabolic programming of distinct cancer stem cells promotes metastasis of pancreatic ductal adenocarcinoma. Oncogene 40 (1), 251-231, 2021. PMID33110235. * both are corresponding authors
Nimmakayala R K, Seshacharyulu P, Lakshmanan I, Rachagani S, Chugh S, Karmakar S, Rauth S, Vengoji R, Atri P, Talmon G A, Lele S M, Smith L M, Thapa I, Bastola D, Ouellette M M, Batra S K*, and Ponnusamy M P*, Cigarette Smoke Induces Stem Cell Features of Pancreatic Cancer Cells via PAFI. Gastroenterology 155 (3): 892-908, 2018. PMID: 29864419. * both are corresponding authors
O'Brien P C, Fleming T R. A multiple testing procedure for clinical trials. Biometrics. 1979; 35 (3): 549-56. PMID: 497341.
Peh H Y, Tan W S, Liao W, Wong W S. Vitamin E therapy beyond cancer: Tocopherol versus tocotrienol. Pharmacol Ther. 2016; 162:152-69. PMID: 26706242.
Permuth J B, Chen D T, Yoder S J, Li J, Smith A T, Choi J W, Kim J, Balagurunathan Y, Jiang K, Coppola D, Centeno B A, Klapman J, Hodul P, Karreth F A, Trevino J G, Merchant N, Magliocco A, Malafa M P, Gillies R. Linc-ing Circulating Long Non-coding RNAs to the Diagnosis and Malignant Prediction of Intraductal Papillary Mucinous Neoplasms of the Pancreas. PMCID: PMC5585319
Permuth J B, Chen D T, Yoder S J, Li J, Smith A T, Choi J W, Kim J, Balagurunathan Y, Jiang K, Coppola D, Centeno B A, Klapman J, Hodul P, Karreth F A, Trevino J G, Merchant N, Magliocco A, Malafa M P, Gillies R. Linc-ing Circulating Long Non-coding RNAs to the Diagnosis and Malignant Prediction of Intraductal Papillary Mucinous Neoplasms of the Pancreas. Sci Rep. 2017; 7 (1): 10484. PMID: 28874676 PMCID: PMC5585319.
Permuth J B, Dezsi K B, Vyas S, Ali K N, Basinski T L, Utuama O A, Denbo J W, Klapman J, Dam A, Carballido E, Kim D W, Pimiento J M, Powers B D, Otto A K, Choi J W, Chen D T, Teer J K, Beato F, Ward A, Cortizas E M, Whisner S Y, Williams I E, Riner A N, Tardif K, Velanovich V, Karachristos A, Douglas W G, Legaspi A, Allan B J, Meredith K, Molina-Vega M A, Bao P, St Julien J, Huguet K L, Green L, Odedina F T, Kumar N B, Simmons V N, George T J, Vadaparampil S T, Hodul P J, Arnoletti J P, Awad Z T, Bose D, Jiang K, Centeno B A,
Gwede C K, Malafa M, Judge S M, Judge A R, Jeong D, Bloomston M, Merchant N B, Fleming J B, Trevino J G, On Behalf Of The Florida Pancreas C. The Florida Pancreas Collaborative Next-Generation Biobank: Infrastructure to Reduce Disparities and Improve Survival for a Diverse Cohort of Patients with Pancreatic Cancer. Cancers (Basel). 2021; 13 (4). PMID: 33671939 PMCID: PMC7919015.
Permuth J B, Georgeades C, Malafa M. MiRNAs as biomarkers of high-risk pancreatic cysts: a possible
Permuth J B, Trevino J, Merchant N, Malafa M, Florida Pancreas C. Partnering to advance early detection
Permuth-Wey J, Chen D T, Fulp W J, Yoder S J, Zhang Y, Georgeades C, Husain K, Centeno B A, Magliocco A M, Coppola D, Malafa M. Plasma MicroRNAs as Novel Biomarkers for Patients with Intraductal Papillary Mucinous Neoplasms of the Pancreas. Cancer Prev Res (Phila). 2015 Sep; 8 (9): 826-34. doi: 10.1158/1940-6207.CAPR-15-0094. PMCID: PMC4560649
Pocock S J. Allocation of patients to treatment in clinical trials. Biometrics. 1979; 35 (1): 183-97. PMID: 497334.
Ponnusamy M. P, Deb S, Dey P, Chakraborty S, Rachagani S, Senapati S, and Batra S K RNA Polymerase I I Associated Factor 1/PD2 Maintains Self-Renewal by Its Interaction with Oct3/4 in Mouse Embryonic Stem Cells. Stem Cells., 27:3001-3011, 2009 PMID: 19821493
Prasad K. Tocotrienols and cardiovascular health. Curr Pharm Des. 2011; 17 (21): 2147-54. PMID: 21774782.
R. Sasson and Batra S K A novel interaction of galectin-3 with MUC4 in pancreatic adenocarcinoma: potential pathological implication. Clinical Cancer Research, 17 (2): 267-74, 2011 PMID21059814.
Rachagani S, Macha MA, Ponnusamy M P, Haridas D, Kaur S, Jain M, Batra S K. MUC4 potentiates invasion and metastasis of pancreatic cancer cells through stabilization of fibroblast growth factor receptor 1. Carcinogenesis. 2012; 33 (10): 1953-64. PMID: 22791819 PMCID: PMC3529557.
Rahib L, Smith B D, Aizenberg R, Rosenzweig A B, Fleshman J M, Matrisian L M. Projecting cancer incidence and deaths to 2030: the unexpected burden of thyroid, liver, and pancreas cancers in the United States. Cancer Res. 2014; 74 (11): 2913-21. PMID: 24840647.
reverses chemoresistance of pancreatic cancer stem/progenitor cells and their progenies. Cancer Lett. 2010; 295 (1): 69-84. PMID: 20303649 PMCID: PMC2891434.
Ricci C, Migliori M, Imbrogno A, Mazzotta E, Felicani C, Serra C, Bergonzoni B, Calculli L, Casadei R. Prevalence of Asymptomatic Intraductal Papillary Mucinous Neoplasms in Healthy and Ill Populations Detected by Ultrasonography: A Single-Center Study of 6353 Outpatients. Pancreas. 2019; 48 (1): 113-20. PMID: 30451793.
Senapati S, Gnanapragassam V S, Moniaux N, Momi N, Batra S K. Role of MUC4-NIDO domain in the MUC4-mediated metastasis of pancreatic cancer cells. Oncogene. 31 (28): 3346-56, 2012
Senapati S., Chaturvedi P., Chaney W. Subhankar Chakraborty, Vinayaga S. Gnanapragassam, Aaron
Siegel R L, Miller K D, Jemal A. Cancer statistics, 2020. CA Cancer J Clin. 2020; 70 (1): 7-30. PMID: 31912902.
Singh A P, Chaturvedi P, Batra S K. Emerging roles of MUC4 in cancer: a novel target for diagnosis and therapy. Cancer Res. 2007; 67 (2): 433-6. PMID: 17234748.
Singh A P, Moniaux N, Chauhan S C, Meza J, and Batra S K. Inhibition of MUC4 Expression suppresses pancreatic tumor cell growth and metastasis. Cancer Research, 64, 622-630, 2004. PMID14744777
Singhi A D, Nikiforova M N, Fasanella K E, McGrath K M, Pai R K, Ohori N P, Bartholow T L, Brand R E, Chennat J S, Lu X, Papachristou G I, Slivka A, Zeh H J, Zureikat A H, Lee K K, Tsung A, Mantha G S, Khalid A. Preoperative GNAS and KRAS testing in the diagnosis of pancreatic mucinous cysts. Clin Cancer Res. 2014; 20 (16): 4381-9. PMID: 24938521.
Springett G M, Husain K, Neuger A, Centeno B, Chen D T, Hutchinson T Z, Lush R M, Sebti S, Malafa M P. A Phase I Safety, Pharmacokinetic, and Pharmacodynamic Presurgical Trial of Vitamin E delta-tocotrienol in Patients with Pancreatic Ductal Neoplasia. EBioMedicine. 2015; 2 (12): 1987-95. PMID: 26844278 PMCID: PMC4703733.
Springett G M. Husain K, Neuger A, Centeno B, Chen D T, Hutchinson T Z, Lush R M, Sebti S, Malafa M P. A Phase I Safety, Pharmacokinetic, and Pharmacodynamic Presurgical Trial of Vitamin E o-tocotrienol in Patients with Pancreatic Ductal Neoplasia. EBioMedicine. 2015 Nov. 14; 2 (12): 1987-95. PMCID: PMC4703733
Stan S D, Singh S V, Brand R E. Chemoprevention strategies for pancreatic cancer. Nat Rev Gastroenterol Hepatol. 2010; 7 (6): 347-56. PMID: 20440279 PMCID: PMC2927967.
Takenaka A. Development of ductal carcinoma of the pancreas during follow-up of branch duct intraductal papillary mucinous neoplasm of the pancreas. Gut. 2008; 57 (11): 1561-5. PMID: 18477671.
Tanaka M, Fernandez-Del Castillo C, Kamisawa T, Jang J Y, Levy P, Ohtsuka T, Salvia R, Shimizu Y, Tada M, Wolfgang C L. Revisions of international consensus Fukuoka guidelines for the management of IPMN of the pancreas. Pancreatology. 2017; 17 (5): 738-53. PMID: 28735806.
Tanno S, Obara T, Koizumi K, Nakano Y, Osanai M, Mizukami Y, Kohgo Y. Risk of additional pancreatic cancer in patients with branch duct intraductal papillary-mucinous neoplasm. Clin J Gastroenterol. 2009; 2 (6): 365-70. PMID: 26192788.
Timeline of Progression of Presumed Low-Risk Intraductal Papillary Mucinous Neoplasms. AJR Am J Roentgenol. 2017; 209 (2): 320-6. PMID: 28590817.
Trapnell C, Hendrickson D G, Sauvageau M, Goff L, Rinn J L, Pachter L. Differential analysis of gene regulation at transcript resolution with RNA-seq. Nat Biotechnol. 2013; 31 (1): 46-53. PMID: 23222703 PMCID: PMC3869392.
Tsai J H, Liau J Y, Yuan C T, Cheng M L, Yuan R H, Jeng Y M. RNF43 mutation frequently occurs with GNAS mutation and mucin hypersecretion in intraductal papillary neoplasms of the bile duct. Histopathology. 2017; 70 (5): 756-65. PMID: 27864998.
Uehara H, Nakaizumi A, Ishikawa O, Jishi H, Tatsumi K, Takakura R, Ishida T, Takano Y, Tanaka S,
Wang C, Husain K, Zhang A, Centeno B A, Chen D T, Tong Z, Sebti S M, Malafa M P. EGR-1/Bax pathway plays a role in vitamin E delta-tocotrienol-induced apoptosis in pancreatic cancer cells. J Nutr Biochem. 2015; 26 (8): 797-807. PMID: 25997867 PMCID: PMC4576995.
Wang C, Husain K, Zhang A, Centeno B A, Chen D T, Tong Z, Sebti S M, Malafa M P. EGR-1/Bax pathway plays a role in vitamin E delta-tocotrienol-induced apoptosis in pancreatic cancer cells. J Nutr Biochem. 2015; 26 (8): 797-807. PMID: 25997867 PMCID: PMC4576995.
Wu J, Matthaei H, Maitra A, Dal Molin M, Wood L D, Eshleman J R, Goggins M, Canto M I, Schulick R D, Edil B H, Wolfgang C L, Klein A P, Diaz L A, Jr., Allen P J, Schmidt C M, Kinzler K W, Papadopoulos N, Hruban R H, Vogelstein B. Recurrent GNAS mutations define an unexpected pathway for pancreatic cyst development. Sci Transl Med. 2011; 3 (92): 92ra66. PMID: 21775669 PMCID: PMC3160649.
Yang C S, Luo P, Zeng Z, Wang H, Malafa M, Suh N. Vitamin E and cancer prevention: Studies with

1. A method of inhibiting progression of a neoplasm to a cancer in a subject comprising administering to the subject a therapeutically effective amount of a composition comprising δ-tocotrienol (d-T3).

2. The method of inhibiting progression of a cancer of claim 1, wherein the cancer progression being inhibiting is progression occurring following surgical removal or anti-cancer treatment of a cancer.

3. The method of claim 1, wherein the cancer is lung cancer, ovarian cancer, breast cancer, rectal cancer, pancreatic, colon cancer.

4. The method of claim 1 wherein the neoplasm comprises intraductal papillary mucinous neoplasm (IPMN) and the cancer to which progression is being inhibited comprises pancreatic ductal adenocarcinoma.

5. The method of claim 1, further comprising administering to the subject aspirin.

6. The method of claim 1, wherein the daily dose of d-T3 is between about 400 mg and 800 mg.

7. The method of claim 1, wherein the composition comprises 400 mg of d-T3.

8. The method of claim 1, wherein the d-T3 composition is administered twice daily.

9. The method of claim 1, wherein the d-T3 composition is administered for 6 months.

10. The method of claim 1, wherein the d-T3 composition is administered orally.
